SEQUENCE LISTING (3 sequences)

SEQ ID NO: 1 - protein, 176 aa, synthetic construct
  REGION 1..176: note = Synthetic peptide, sonic hedgehog (SHH) agonist
IVIGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRALDITTSDRDRSKYGMLARLAVEAGFDWVYYESKAHIHCSVKAENSVAAKSGG

SEQ ID NO: 2 - protein, 175 aa, synthetic construct
  REGION 1..175: note = Synthetic peptide, sonic hedgehog (SHH) agonist C24II
IIGPGRGFGKRRHPKKLTPLAYKQFIPNVAEKTLGASGRYEGKISRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGG

SEQ ID NO: 3 - protein, 175 aa, synthetic construct
  REGION 1..175: note = Synthetic peptide, sonic hedgehog (SHH) agonist C25II
IIGPGRGFGKRRHPKKLTPLAYKQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYESKAHIHCSVKAENSVAAKSGG

(19) United States
(12) Patent Application Publication
(10) Pub. No.: US 2025/0136934 A1
(43) Pub. Date: May 1, 2025

(54) PRODUCTION OF INDUCED MIDBRAIN DOPAMINERGIC PROGENITOR CELLS FROM PLURIPOTENT STEM CELLS

(71) Applicant: NUWACELL BIOTECHNOLOGIES CO., LTD., Hefei (CN)

(72) Inventors: Junying YU, Hefei, Anhui (CN); Luyan JIAO, Hefei, Anhui (CN); Yongqiang ZHAO, Hefei, Anhui (CN); Ying ZHANG, Hefei, Anhui (CN)

(73) Assignee: NUWACELL BIOTECHNOLOGIES CO., LTD., Hefei (CN)

(21) Appl. No.: 18/630,498

(22) Filed: Apr. 9, 2024

Related U.S. Application Data
(63) Continuation of application No. PCT/CN2023/128996, filed on Nov. 1, 2023.

(51) Int. Cl. C12N 5/0793

(52) U.S. Cl. CPC C12N 5/0619; C12N 2500/90; C12N 2500/98; C12N 2501/15; C12N 2501/155; C12N 2501/41; C12N 2501/415; C12N 2501/727; C12N 2506/45

(57) ABSTRACT

The present disclosure is directed to a method of producing induced midbrain dopaminergic progenitor (imDAP) cells which can provide high efficiency for differentiation of imDAP cells. The present disclosure also provides a substantially homogeneous population of imDAP cells.

### CROSS REFERENCE TO RELATED APPLICATIONS

This patent application is a continuation of and claims the benefit of international patent application No. PCT/CN2023/128996, filed on Nov. 1, 2023, the disclosure of which is incorporated herein in its entirety by reference.

### TECHNICAL FIELD

The present disclosure generally relates to the field of stem cell technology, in particular to a method for producing induced midbrain dopaminergic progenitor (imDAP) cells from pluripotent stem cells and derived imDAP cells.

### SEQUENCE LISTING

The contents of the text file submitted electronically herewith are incorporated herein by reference in their entirety: a compute readable format copy of the Sequence Listing (File name: “42231WO_SequenceListing.xml”; Date recorded: Oct. 24, 2023; File size: 5,007 bytes).

### BACKGROUND

Parkinson's disease (PD) is the second most common neurodegenerative disorder. A hallmark of the disease is the selective loss of dopaminergic neurons (DA neurons) of the substantia nigra of the midbrain. Although recent developments in medical science have greatly advanced the general understanding of the pathogenesis of PD, unfortunately, there are no cures for this devastating disease at present. The main treatment for PD patients is DA analogues and receptor agonists to counteract the reductions in DA neurons.

There remains a need to further advance the study for the mechanism of PD, its disease progression, and effective clinical intervention approaches to effectively treat PD, and one pre-requisite for such studies is the availability of midbrain dopaminergic progenitor cells which can be further expanded and matured to generate DA neurons.

Pluripotent stem cells comprise human pluripotent stem cells (hPSCs), including human embryonic stem cells (hESCs) and human induced pluripotent stem cells (hiPSCs). These pluripotent stem cells can be expanded in vitro and retain their capacity to differentiate into any cell types of the three germ layers, including neuronal cells and tissues. Thus, these pluripotent stem cells are invaluable to study developmental processes and disease mechanisms, especially in the brain. In particular, hiPSCs represent an unlimited source of cells for utilities such as mechanism studies, drug screening assays, and eventually cell replacement therapy for the treatment of neurological disorders, such as PD. Numerous protocols have been developed to generate imDAP cells in vitro from hPSCs.

These protocols typically rely on the directed differentiation of pluripotent stem cells into imDAP cells using small molecules and growth factors. They are less efficient and highly variable between product batches, resulting in heterogeneous population with relatively low numbers of imDAP cells. However, in clinical and therapeutical applications, homogeneous and robust populations of cells are strongly desired.

There remains a need to provide an improved method for producing imDAP cells.

### SUMMARY OF THE DISCLOSURE

This disclosure provides a unique differentiation process for generating induced midbrain dopaminergic progenitor (imDAP) cells from pluripotent stem cells. The unique process disclosed herein includes a multistep WNT signaling activation process comprising multiple WNT signaling activation substeps each using a WNT signaling pathway activator, wherein the WNT signaling pathway activator is used at a roller-coaster-like concentration in the multistep WNT signaling activation process. The unique process disclosed herein is highly efficient in the differentiation and well consistent between product batches, resulting in a homogenous and robust cell population with a high percentage of FOXA2+ imDAP cells. imDAP cells derived from the unique differentiation process herein can be expanded, matured, cryopreserved and/or enriched. This disclosure also relates to, among other things, cell populations, cell lines, and/or clonal cells generated using the processes described herein.

One aspect of the present disclosure is directed to a method for producing induced midbrain dopaminergic progenitor (imDAP) cells, comprising: culturing pluripotent stem cells (PSCs) to form embryonic bodies (EBs); and differentiating the EBs into imDAP cells by a multistep WNT signaling activation process, thereby obtaining a cell population comprising imDAP cells, wherein the multistep WNT signaling activation process comprises:
(i) contacting the EBs with a first differentiation medium comprising a WNT signaling pathway activator at a first concentration,
(ii) contacting the cells from step (i) with a second differentiation medium comprising the WNT signaling pathway activator at a second concentration,
(iii) contacting the cells from step (ii) with a third differentiation medium comprising the WNT signaling pathway activator at a third concentration, and
(iv) contacting the cells from step (iii) with a fourth differentiation medium comprising the WNT signaling pathway activator at a fourth concentration,
wherein the first and third concentrations are lower than the fourth concentration, and the second concentration is higher than the fourth concentration.

In some embodiments, the first and third concentrations of the WNT signaling pathway activator are each independently in the range from about 0.2 μM to about 1.6 μM.

In some embodiments, the second concentration of the WNT signaling pathway activator is in the range from about 4 μM to about 10 μM.

In some embodiments, the fourth concentration of the WNT signaling pathway activator is in the range from about 2 μM to about 3.5 μM.

In some embodiments, the WNT signaling pathway activator in each of the first to fourth differentiation media comprises a glycogen synthase kinase-3 (GSK3) inhibitor, e.g., CHIR99021.

In some embodiments, the first differentiation medium further comprises a BMP4 inhibitor, a TGF-β inhibitor, and a sonic hedgehog agonist, and the second to fourth differentiation media each further comprise none, one or more of a BMP4 inhibitor, a TGF-β inhibitor, and a sonic hedgehog agonist.

In some embodiments, the concentration of the BMP4 inhibitor, if present in the first, second, third, or fourth differentiation medium, is in the range from about 0.05 μM to about 2.0 μM.

In some embodiments, the BMP4 inhibitor is Noggin, Chordin, Follostatin, Dorsomorphin, LDN193189, or any combination thereof.

In some embodiments, the concentration of the TGF-β inhibitor, if present in the first, second, third, or fourth differentiation medium, is in the range from about 1 μM to about 100 μM.

In some embodiments, the TGF-β inhibitor is RepSox, A83-01, SB431542, D4476, GW788388, LY364947, LY580276, SB525334, SB505124, SD208, GW6604, SJN-2511, or any combination thereof.

In some embodiments, the concentration of the sonic hedgehog agonist, if present in the first, second, third, or fourth differentiation medium, is in the range from about 100 ng/ml to about 2000 ng/mL.

In some embodiments, the sonic hedgehog agonist is SHH, SHH C24II, SHH C25II, SAG, SMO-IN-1, purmorphamine, an analogue thereof, or any combination thereof.

In some embodiments, the first differentiation medium comprises CHIR99021, LDN193189, SB431542, and SHH C24II, and the second to fourth differentiation media each comprise CHIR99021 and none, one or more of LDN193189, SB431542, and SHH C24II.

In some embodiments, the first differentiation medium comprises CHIR99021, LDN193189, SB431542, SHH C24II, Purmorphamine and SAG, and the second to fourth differentiation media each comprise CHIR99021 and none, one or more of LDN193189, SB431542, SHH C24II, Purmorphamine, and SAG.

In some embodiments, the first to fourth differentiation media further comprise a serum-free and xeno-free neural basal medium.

In some embodiments, the neural basal medium in the first to fourth differentiation media comprises an ascorbic compound, e.g., L-ascorbic acid 2-phosphate sesquimagnesium salt.

In some embodiments, the neural basal medium is free of vitamin A.

In some embodiments, the concentrations of the ascorbic compound in the first to fourth differentiation media each are about 5 μg/mL to about 100 μg/mL.

In some embodiments, the duration for step (i) is about 3 to about 4 days.

In some embodiments, the duration for step (ii) is about 2 to about 5 days.

In some embodiments, the duration for step (i) is about 4 days and the duration for step (ii) is about 2 to about 4 days.

In some embodiments, the duration for step (iii) is about 2 to about 5 days.

In some embodiments, the duration for step (iv) is about 2 to about 6 days.

In some embodiments, the time window for performing the step (i) is from Day 0 to Day 4 and the time window for performing the step (ii) is from Day 4 to Day 6, from Day 4 to Day 7, or from Day 4 to Day 8.

In some embodiments, the time window for performing the step (i) is from Day 0 to Day 4 and the time window for performing the step (ii) is from Day 4 to Day 8.

In some embodiments, the timing for treatment of the BMP inhibitor in the multistep WNT signaling activation process is from Day 0 to Day 6 and the timing for treatment of the TGF-β inhibitor in the multistep WNT signaling activation process is from Day 0 to Day 4.

In some embodiments, the concentration of the BMP inhibitor is 0.5 μM, and the concentration of the TGF-β inhibitor is 20 μM.

In some embodiments, the concentration of the BMP inhibitor is 0.1 μM, and the concentration of the TGF-β inhibitor is 50 μM.

In some embodiments, the concentration of the BMP inhibitor is 0.2 μM, the concentration of the TGF-β inhibitor is 50 μM, the timing for treatment of the BMP inhibitor in the multistep WNT signaling activation process is from Day 0 to Day 6, and the timing for treatment of the TGF-β inhibitor in the multistep WNT signaling activation process is from Day 0 to Day 4, or from Day 0 to Day 6.

In some embodiments, the concentration of the BMP inhibitor is 0.5 μM, the concentration of the TGF-β inhibitor is 20 μM, the timing for treatment of the BMP inhibitor in the multistep WNT signaling activation process is from Day 0 to Day 6, or from Day 0 to Day 8, and the timing for treatment of the TGF-β inhibitor in the multistep WNT signaling activation process is from Day 0 to Day 4.

In some embodiments, the method further comprises expanding the imDAP cells after differentiation.

In some embodiments, the imDAP cells are expanded for one passage after differentiation.

In some embodiments, the imDAP cells are expanded at a seeding density of about 1×10⁴ cells/cm² to about 3×10⁴ cells/cm².

In some embodiments, the method further comprises enriching the imDAP cells by sorting for TPBG+ cells after differentiation.

In some embodiments, the method further comprises enriching the imDAP cells by sorting for TPBG+ cells after expansion for one passage.

In some embodiments, after differentiation, at least about 78%, preferably at least about 85%, more preferably at least 90%, and most preferably 95% to 100% of the cells in the cell population without any enrichment are FOXA2+ imDAP cells.

In some embodiments, after differentiation, at least about 60%, preferably from about 60% to about 90%, more preferably about 70% to about 90%, and most preferably 80% to 90% of the cells in the cell population without any enrichment are FOXA2+OTX2+ imDAP cells.

In some embodiments, after differentiation, at least about 50%, preferably from about 50% to about 90%, more preferably about 50% to about 80%, and most preferably 60% to 80% of the cells in the cell population without any enrichment are LMX1A+ imDAP cells.

In some embodiments, after enrichment, at least about 90%, preferably from about 95%, more preferably at least about 98% and most preferably 100% of the cells in the cell population obtained are LMX1A+ imDAP cells.

In some embodiments, after enrichment, at least about 90%, preferably from about 95%, more preferably at least about 98% and most preferably 100% of the cells in the cell population obtained are OTX2+ imDAP cells.

In some embodiments, the imDAP cells are FOXA2+OTX2+EN1+LMX1A+PAX6−NKX2.1− cells.

Another aspect of the disclosure is directed to a population of imDAP cells prepared by the method disclosed herein.

Various objects and advantages of the reagents, compositions and methods as provided herein will become apparent from the following description taken in conjunction with the accompanying drawings wherein are set forth, by way of illustration and example, certain embodiments of the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: An exemplary schematic diagram of an example of the method for producing imDAP cells from PSCs according to this disclosure.

FIG. 2: qRT-PCR analysis of the expressions of EN1 and LMX1A for 3 batches of imDAP cells derived from the 2-step WNT signaling activation protocol.

FIG. 3: qRT-PCR analysis of the expressions of EN1 and LMX1A for imDAP cells derived from the 3-step WNT signaling activation protocol where high concentrations of CHIR at 5 μM, 7.5 μM and 10 μM (Boost CHIR) were used, respectively.

FIG. 4: qRT-PCR analysis of the expressions of EN1 and LMX1A for 3 batches of imDAP cells derived from the 3-step WNT signaling activation protocol.

FIG. 5: FACS analysis of FOXA2 expression among 3 batches of imDAP cells derived from the 3-step WNT signaling activation protocol.

FIG. 6: qRT-PCR analysis of the expressions of EN1 and LMX1A for imDAP cells derived from the 3-step WNT signaling activation protocol where different time windows for the Stage 2-2 are used, respectively.

FIG. 7: FACS analysis of FOXA2 expression for imDAP cells derived from the 4-step WNT signaling activation protocol with different SHH agonists.

FIG. 8A: FACS analysis of OTX2 expression for imDAP cells derived from the 4-step WNT signaling activation protocol where different time windows for the Stage 2-2 are used, respectively.

FIG. 8B: qRT-PCR analysis of the expression of dorsal midbrain marker PAX6 for imDAP cells derived from the 4-step WNT signaling activation protocol where different time windows for the Stage 2-2 are used, respectively.

FIG. 9: FACS analysis of FOXA2 expression for imDAP cells derived from the 4-step WNT signaling activation protocol where low CHIR concentration at 1.2 μM, 1.4 μM and 1.6 μM were used in the Stage 2-3, respectively.

FIG. 10A: FACS analysis of FOXA2+/OTX2+ double positive expression for 3 batches of imDAP cells derived from the 4-step WNT signaling activation protocol.

FIG. 10B: qRT-PCR analysis of the expressions of EN1 and LMX1A for 3 batches of imDAP cells derived from the 4-step WNT signaling activation protocol.

FIG. 11A: FACS analysis of FOXA2+/OTX2+ double positive expression for imDAP cells derived from the 4-step WNT signaling activation protocol where different time windows for treatments of LDN193189 and SB431542 are used, respectively.

FIG. 11B: qRT-PCR analysis of the expression of NKX2.1 for imDAP cells derived from the 4-step WNT signaling activation protocol where different time windows for treatments of LDN193189 and SB431542 are used, respectively.

FIG. 11C: Representative immunofluorescent staining of LMX1A (red) and DAPI (blue) for imDAP cells derived from the 4-step WNT signaling activation protocol where different time windows for treatments of LDN193189 and SB431542 are used, respectively. In the Figure, the percentage of LMX1A+ cells are qualitatively indicated by the signs “+” (moderate), “++” (high), and “+++” (very high). Scale bar: 50 μm.

FIG. 12A: FACS analysis of FOXA2+/OTX2+ double positive expression for imDAP cells of Day 6 derived from the 4-step WNT signaling activation protocol where different concentrations of LDN193189 and SB431542 are used, respectively.

FIG. 12B: FACS analysis of FOXA2+/OTX2+ double positive expression for imDAP cells of Day 12 derived from the 4-step WNT signaling activation protocol where different concentrations of LDN193189 and SB431542 are used, respectively.

FIG. 12C: Representative immunofluorescent staining of LMX1A (red) and DAPI (blue) for imDAP cells of Day 12 derived from the 4-step WNT signaling activation protocol where different concentrations of LDN193189 and SB431542 are used, respectively. In the Figure, the percentage of LMX1A+ cells are qualitatively indicated by the signs “+” (moderate), “++” (high), and “+++” (very high). Scale bar: 50 μm.

FIG. 13A: FACS analysis of FOXA2+/OTX2+ double positive expression for imDAP cells derived from the 4-step WNT signaling activation protocol where different time windows for treatments with LDN193189 at 0.2 μM and SB431542 at 50 μM are used, respectively.

FIG. 13B: FACS analysis of FOXA2+/OTX2+ double positive expression for imDAP cells derived from the 4-step WNT signaling activation protocol where different time windows for treatments with LDN193189 at 0.5 μM and SB431542 at 20 μM are used, respectively.

FIG. 13C: Representative immunofluorescent staining of LMX1A (red) and DAPI (blue) for imDAP cells derived from the 4-step WNT signaling activation protocol where different time windows for treatments with LDN193189 at 0.2 μM and SB431542 at 50 μM are used, respectively. In the Figure, the percentage of LMX1A+ cells are qualitatively indicated by the signs “+” (moderate), “++” (high), and “+++” (very high). Scale bar: 50 μm.

FIG. 13D: Representative immunofluorescent staining of LMX1A (red) and DAPI (blue) for imDAP cells derived from the 4-step WNT signaling activation protocol where different time windows for treatments with LDN193189 at 0.5 μM and SB431542 at 20 μM are used, respectively. In the Figure, the percentage of LMX1A+ cells are qualitatively indicated by the signs “+” (moderate), “++” (high), and “+++” (very high). Scale bar: 50 μm.

FIG. 14: FACS analysis of FOXA2 expression for imDAP cells derived from the 4-step WNT signaling activation protocol where the neural basal medium with or without the addition of L-ascorbic acid 2-phosphate sesquimagnesium salt and/or vitamin A is used in the first to fourth differentiation media, respectively. In the Figure, “w/o ascorbic acid” represents the case where L-ascorbic acid 2-phosphate sesquimagnesium salt is not added throughout the process of differentiation, “w/ascorbic acid” represents the case where L-ascorbic acid 2-phosphate sesquimagnesium salt is added throughout the process of differentiation, “VitA D0-3” represents the case where vitamin A is added only in the initial 3 days of differentiation, “VitA D0-2” represents the case where vitamin A is added only in the initial 2 days of differentiation, and “w/o VitA” represents the case where vitamin A is not added throughout the process of differentiation.

FIG. 15A: FACS analysis of TPBG expression for imDAP cells of Day 6 (D6) and Day 12 (P0) differentiated from the 4-step WNT signaling activation protocol as well as imDAP cells expanded for 1 passage (P1) and 2 passages (P2) from the differentiated imDAP cells (P0).

FIG. 15B: FACS analysis of TPBG expression for imDAP cells of P0 and P1 before and after enrichment.

FIG. 15C: Representative immunofluorescent staining (with DAPI staining (nucleus, blue)) of the expression of LMX1A (red) for imDAP cells of P0 and P1 before and after enrichment. Scale bar: 50 μm.

FIG. 15D: Quantification of the percentage of LMX1A expression for imDAP cells of P0 and P1 before and after enrichment.

FIG. 16A: FACS analysis of TPBG expression for cells of P1 expanded from imDAP cells of P0 at different seeding density (shown by days required to reach 100% confluence) before and after enrichment.

FIG. 16B: Sorting recovery for cells of P1 expanded from imDAP cells of P0 at different seeding density (shown by days required to reach 100% confluence) after enrichment.

FIG. 16C: Representative immunofluorescent images (with DAPI staining (nucleus, blue)) for the expressions of LMX1A (red) and OTX2 (green) for imDAP cells of P1 (pre-sorting) and P3 (post-sorting). Scale bar: 50 μm.

FIG. 16D: Quantification of the percentages of LMX1A and OTX2 expressions for imDAP cells of P1 (pre-sorting) and P3 (post-sorting).

### DETAILED DESCRIPTION

It is to be appreciated that certain aspects, modes, embodiments, variations, and features of the present disclosure are described below in various levels of detail in order to provide a substantial understanding of the present technology.

Reference throughout this specification to “first,” “second,” “third,” “fourth,” “fifth,” “sixth,” “seventh,” “eighth,” or “ninth” does not mean the order or sequence of the feature, structure (e.g., medium or composition) or characteristic described in connection with the reference and can be used only for the purpose of distinction.

Reference throughout this specification to “a first aspect,” “a second aspect,” “a third aspect,” “a fourth aspect,” “a fifth aspect,” “a sixth aspect,” “a seventh aspect,” “an eighth aspect,” or “a ninth aspect” means that a particular feature, structure or characteristic described in connection with the aspect is included in at least one or more aspects of the present disclosure. Also, the particular feature(s), structure(s), characteristic(s) or embodiment(s) in one aspect may be combined with those in one or more other aspects in any suitable manner.

Reference throughout this specification to “one embodiment,” “some embodiments,” “a preferred embodiment(s),” “certain embodiments” or “a certain embodiment(s)” means that a particular feature, structure or characteristic described in connection with the embodiment(s) is included in at least one or more embodiments of the present disclosure. Also, the particular feature(s), structure(s), or characteristic(s) in one embodiment may be combined with those in one or more other embodiments in any suitable manner.

It is to be understood that the present disclosure is not limited to particular uses, methods, reagents, compounds, compositions or biological systems, which can, of course, vary. It is also to be understood that the terminology used herein is for the purpose of describing particular embodiments only and is not intended to be limiting.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd cd. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

Unless otherwise specified, “a” or “an” means “one or more.”

As used herein, “about” means plus or minus 10%, or plus or minus 5%, or plus or minus 4%, or plus or minus 3%, or plus or minus 2%, or plus or minus 1%, as well as the specified number.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of). Further, in each instance herein any of the terms “comprising,” “consisting essentially of,” and “consisting of” may be replaced with either of the other two terms.

As used herein, the term “pluripotent stem cell” (PSC) refers to cells that have the capability to self-renew in an undifferentiated state and to differentiate into almost any cell type in the body. Pluripotent stem cells can be pluripotent and give rise during development to all derivatives of the three primary germ layers: ectoderm, endoderm and mesoderm. Pluripotent stem cells can be of human origin (e.g., human PSC or hPSC). Pluripotent stems cells can be induced pluripotent stem cells (iPSCs) or embryonic stem cells (ESCs). In some embodiments, the pluripotent stem cells are human induced pluripotent stem cells (hiPSCs). ESCs (e.g., hESCs) and iPSCs (e.g., hiPSCs) are known in the art and can be readily obtained using conventional methods, for example, those described in the existing technologies, or commercially available products. PSCs obtained following various types of genetic engineering, such as transgene knock-in can also be used herein.

As used herein, the term “embryonic stem cells,” or “ESCs” refers to naturally occurring pluripotent stem cells of the inner cell mass of the embryonic blastocyst. Embryonic stem cells are pluripotent and give rise during development to all derivatives of the three primary germ layers: ectoderm, endoderm and mesoderm. They do not contribute to the extraembryonic membranes or the placenta, i.e., are not totipotent. When used in the present disclosure, the embryonic stem cells or ESCs are sourced from commercially established human embryonic stem cell lines or human embryonic stem cells isolated or acquired from early embryos that have developed in vitro for not more than 14 days from fertilization.

As used herein, the term “induced pluripotent stem cells” or, iPSCs, means that the stem cells are produced from differentiated adult, neonatal or fetal cells that have been induced or changed, i.e., reprogrammed into cells capable of differentiating into tissues of all three germ or dermal layers: mesoderm, endoderm, and ectoderm. The iPSCs produced do not refer to cells as they are found in nature. Suitable methods for the generation of iPSCs from somatic or multipotent stem cells are well known to those of skill in the art. For example, iPSCs may be reliably generated from somatic cells by conventional reprogramming technologies. For example, a method for reprogramming erythrocyte progenitor cells to generate hiPSCs has been described in detail in CN108373998B, which is owned by the present applicant and the disclosure of which is incorporated herein by reference in its entirety.

As used herein, the term “pluripotency” or “pluripotent” refers to the developmental potential of a cell to differentiate into cells of all three germ layers (Ectoderm, mesoderm, and endoderm). Pluripotency can be determined, at least in part, by assessing pluripotency characteristics of the cells. Pluripotency characteristics include, but are not limited to: (i) pluripotent stem cell morphology; (ii) the potential for unlimited self-renewal; (iii) expression of pluripotent stem cell markers including, but not limited to SSEA1 (mouse only), SSEA3/4, SSEA5, TRA1-60/81, TRA1-85, TRA2-54, GCTM-2, TG343, TG30, CD9, CD29, CD133/prominin, CD140a, CD56, CD73, CD90, CD105, OCT4, NANOG, SOX2, CD30 and/or CD50; (iv) ability to differentiate to all three somatic lineages (ectoderm, mesoderm and endoderm); (v) teratoma formation consisting of the three somatic lineages; and (vi) formation of embryoid bodies consisting of cells from the three somatic lineages.

As used herein, the term “pluripotent stem cell morphology” refers to the classical morphological features of an embryonic stem cell. Normal embryonic stem cell morphology can be characterized as small and round in shape, with a high nucleus-to-cytoplasm ratio, the notable presence of nucleoli, and/or typical inter-cell spacing.

As used herein, the term “reprogramming” refers to a method of increasing the potency of a cell or dedifferentiating a cell to a less differentiated state. For example, a cell that has an increased cell potency can have more developmental plasticity (i.e., can differentiate into more cell types) compared to the same cell in the non-reprogrammed state. That is, a reprogrammed cell is one that is in a less differentiated state than the same cell in a non-reprogrammed state. “Reprogramming” can refer to de-differentiating a somatic cell, or a multipotent stem cell, into a pluripotent stem cell, also referred to as an induced pluripotent stem cell, or iPSC.

As used herein, the term “differentiation” refers to the process by which an unspecialized (“uncommitted”) or less specialized cell acquires the features of a specialized cell such as, for example, a blood cell or an immune cell. In certain embodiments, a differentiated or differentiation-induced cell is one that has taken on a more specialized (“committed”) position within the lineage of a cell. For example, a human Pluripotent Stem Cell (hPSCs) can be differentiated into various more differentiated cell types, for example, a neural progenitor cell (e.g., midbrain dopaminergic progenitor), a hematopoietic progenitor cell, a lymphocyte, a cardiomyocyte, an immune cell, and other cell types, upon treatment with suitable differentiation factors in the cell culture medium. In certain embodiments, the term “committed” is applied to the process of differentiation to refer to a cell that has proceeded through a differentiation pathway to a point where, under normal circumstances, it would or will continue to differentiate into a specific cell type or subset of cell types, and cannot, under normal circumstances, differentiate into a different cell type (other than a specific cell type or subset of cell types) nor revert to a less differentiated cell type. The term “differentiation” herein is also referred to as “directed differentiation”.

As used herein, the term “midbrain dopaminergic progenitor” (“mDAP”) refers to a neural progenitor cell having ventral midbrain identity. Midbrain dopaminergic progenitors may arise from the ventral mesencephalic area by the combined actions of secreted factors and their downstream transcription factors. These midbrain dopaminergic progenitors proliferate, migrate to their final destinations, and develop into mature midbrain dopaminergic neurons in the substantia nigra and the ventral tegmental area. By the term “ventral midbrain identity” it means that the neural progenitor cells in vitro express markers specific to midbrain and do not express markers specific to the other regional progenitor cells of the brain (i.e. forebrain or hindbrain). Typical positive markers for midbrain dopaminergic progenitor may include FOXA2, OTX2, EN1, and LMX1A. Negative markers for midbrain dopaminergic progenitor may include NKX2.1 and PAX6.

As used herein, the term “induced midbrain dopaminergic progenitor” (imDAP) refers to midbrain dopaminergic progenitor cells generated from pluripotent stem cells in vitro. This disclosure describes a method for generating imDAP cells from induced pluripotent stem cells through a multistep differentiation process. imDAP cells possess the morphological, structural (e.g., markers) and functional characteristics similar to those mDAPs present in vivo. For example, imDAP cells have the potential to develop into mature midbrain dopaminergic neurons and express markers FOXA2, OTX2, EN1, and LMX1A. In some embodiments, the imDAP cells generated by the methods disclosed herein are characterized as FOXA2+OTX2+EN1+LMX1A+PAX6−NKX2.1−.

As used herein, the term “embryoid body” (EB) refers to a three-dimensional cluster of cells that have been shown to mimic embryo development as it gives rise to numerous lineages within its three-dimensional area.

As used herein, the term “culture medium” refers to a culture medium which can support the survival, growth, propagation, maintenance and/or differentiation of cells in an in vitro environment. A culture medium may comprise or have a basal medium and one or more supplements.

As used herein, the term “maintenance culture medium” refers to a culture medium which can support the survival, growth, propagation, or maintenance of cells in an in vitro environment.

As used herein, the term “differentiation culture medium”, “differentiation medium” or “differentiation culture media” refers to a culture medium(s) which can support the differentiation of cells in an in vitro environment. A differentiation culture medium typically comprises a basal medium supplemented with components that induce and/or promote differentiation.

As used herein, the term “basal medium” refers to a basal component of a culture medium (e.g., differentiation culture medium, or expansion culture medium) for cells relative to its supplement(s). Generally, the basal medium comprises about 95% to 99% by volume of the culture medium (e.g., differentiation culture medium, or expansion culture medium). A basal medium of a cell maintenance culture medium acts as a source of nutrients, hormones and/or other factors helpful to propagate and/or sustain the cells. A basal medium of a cell differentiation culture medium acts as a source of nutrients, hormones and/or other factors helpful to differentiate the cells.

As used herein, the term “neural basal medium” refers to a basal component of a culture medium (e.g., differentiation culture medium, or expansion culture medium) for neural cells or neural progenitor cells relative to its supplement(s). Generally, the neural basal medium comprises about 95% to 99% by volume of the culture medium (e.g., differentiation culture medium, or expansion culture medium). A neural basal medium of a maintenance culture medium acts as a source of nutrients, hormones and/or other factors helpful to propagate and/or sustain the neural cells or neural progenitor cells. A neural basal medium of a differentiation culture medium acts as a source of nutrients, hormones and/or other factors helpful to differentiate the neural cells or neural progenitor cells.

As used herein, the term “supplement(s)” refers to an additive component(s) of a culture medium (e.g., differentiation culture medium, or expansion culture medium) relative to its basal medium.

As used herein, the term “supplemented” refers to the addition of a supplement for a culture medium (e.g., differentiation culture medium) into its basal medium. The supplement(s) may be added into a basal medium of a culture medium before or upon the use of the culture medium.

As used herein, the term “in vitro” refers generally to activities that take place outside an organism. As used herein, the term “in vivo” refers generally to activities that take place inside an organism.

As used herein, the term “ex vivo” refers generally to activities that take place outside an organism, such as experimentation or measurements done in or on living tissue in an artificial environment outside the organism, preferably with minimum alteration of the natural conditions. In particular embodiments, “ex vivo” procedures involve living cells or tissues taken from an organism and cultured in a laboratory apparatus, usually under sterile conditions, and typically for a few hours or up to about 24 hours but including up to 48 or 72 hours or longer, depending on the circumstances. In certain embodiments, such tissues or cells can be collected and frozen, and later thawed for ex vivo treatment. Tissue culture experiments or procedures lasting longer than a few days using living cells or tissue are typically considered to be “in vitro,” though in certain embodiments this term can be used interchangeably with ex vivo.

As used herein, the term “cell population” or “population of cells” refers to a group of at least two cells expressing identical, similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing identical, similar or different phenotypes.

As used herein, the term “effective amount” refers to a quantity of an agent sufficient to achieve a beneficial or desired result upon administration.

As used herein, the terms “treatment,” “treat,” and “treating” refer to a clinical intervention aimed to reverse, alleviate, delay the onset of, or inhibit the progress, ameliorate, reduce severity of, prevent or delay the recurrence of a disease, disorder, and/or condition or one or more symptoms thereof, and/or improve one or more symptoms of a disease, disorder, and/or condition as described herein. Treatment, e.g., in the form of imDAP cells or a population of imDAP cells as described herein, may be administered to a subject after one or more symptoms have developed and/or after a disease has been diagnosed. Treatment may be administered in the absence of symptoms, e.g., to prevent or delay onset of a symptom or inhibit onset or progression of a disease. For example, treatment may be administered to a susceptible individual prior to the onset of symptoms (e.g., in light of genetic or other susceptibility factors). Treatment may also be continued after symptoms have resolved, for example to prevent or delay their recurrence. Treatment can result in improvement and/or resolution of one or more symptoms of a disease, disorder and/or condition.

As used herein, the terms “prevent,” “preventing,” and “prevention” refer to reducing the probability of developing a disease, disorder, or condition in a subject, who does not have, but is at risk of or susceptible to developing a disease, disorder, or condition.

Production of Induced Midbrain Dopaminergic Progenitor (imDAP) Cells from Pluripotent Stem Cells (PSCs)

The present disclosure provides a method for producing induced midbrain dopaminergic progenitor (imDAP) cells, comprising: culturing pluripotent stem cells (PSCs) to form embryonic bodies (EBs); and differentiating the EBs into imDAP cells by a multistep WNT signaling activation process, thereby obtaining a cell population comprising imDAP cells, wherein the multistep WNT signaling activation process comprises: (i) contacting the EBs with a first differentiation medium comprising a WNT signaling pathway activator at a first concentration, (ii) contacting the cells from step (i) with a second differentiation medium comprising the WNT signaling pathway activator at a second concentration, (iii) contacting the cells from step (ii) with a third differentiation medium comprising the WNT signaling pathway activator at a third concentration, and (iv) contacting the cells from step (iii) with a fourth differentiation medium comprising the WNT signaling pathway activator at a fourth concentration, wherein the first and third concentrations are lower than the fourth concentration, and the second concentration is higher than the fourth concentration.

It has been uniquely discovered in accordance with this disclosure that the method disclosed herein can provide a more homogeneous and robust cell population with a significantly increased percentage of FOXA2+ imDAP cells and improved batch-to-batch consistency at a higher efficiency for differentiation over the existing methodologies, which is believed to be attributed to the step (iii). In detail, the cell population provided by this method had high percentages for FOXA2 and/or OTX2 expressing cells as well as high expression levels of EN1 and LMX1A, and exhibit superior batch-to batch consistency for the percentage of FOXA2+/OTX2+ cells and the expression level of EN1 and LMX1A among different product batches.

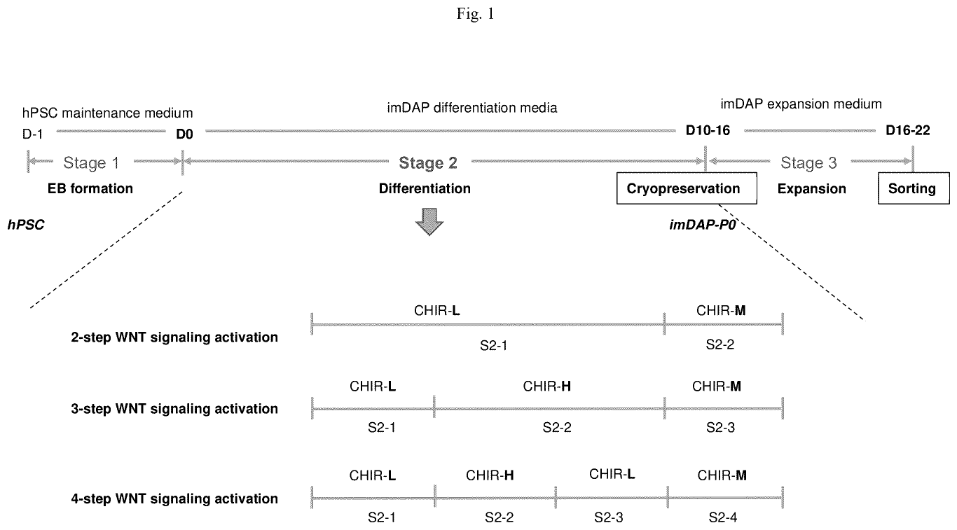

FIG. 1 shows an exemplary graphic representation of an example of the method for producing imDAP cells from PSCs according to this disclosure. As shown in FIG. 1, the method comprises the following stages: Stage 1: EB formation from hiPSCs; Stage 2: differentiation of EBs into imDAP cells; and Stage 3: expansion of imDAP cells. Stage 2 is carried out by a 4-step WNT signaling activation process comprising S2-1 where CHIR is at a low concentration (CHIR-L), S2-2 where CHIR is at a high concentration (CHIR-H), S2-3 where CHIR is at a low concentration (CHIR-L) and S2-4 where CHIR is at an intermediate concentration (CHIR-M). FIG. 1 further shows the 2-step WNT signaling activation process and the 3-step WNT signaling activation process for comparison. It is also shown that the exemplary method further comprises stages of cryopreservation and sorting after Stage 2.

Each step of the method disclosed herein will be described in more details below.

### EB Formation

PSCs can be cultured using a maintenance medium to form EBs. Formation of the EBs from the PSCs can be achieved by suspension maintenance culture, hanging drop EB formation, or Spin EB formation. In preferred embodiments, the EBs are formed by suspension maintenance culture. For example, PSCs can be dissociated to form a single-cell suspension and resuspended in a maintenance culture medium to form embryoid bodies (EBs). The maintenance medium can be for example, Epic medium, E8 or mTeSR or other similar medium. In some embodiments, the maintenance medium contains a ROCK inhibitor. In some embodiments, a concentration range of ROCK inhibitor is from about 2 μM to about 20 μM, preferably from about 4 μM to about 10 μM.

### ROCK Inhibitors

ROCK inhibitor is a compound that targets rho kinase (ROCK) and inhibit the ROCK pathway. Use of ROCK inhibitors improve survival of pluripotent stem cells (PSCs) and formation of EBs from the PSCs.

In certain embodiments, the ROCK inhibitor is selected from the group consisting of: Y-27632, Blebbistatin, HA-100, H-1152, HA-1077, and any combination thereof.

Representative structures of certain ROCK inhibitors that may be used in the maintenance medium of the present disclosure are provided below, many of which are widely commercially available from multiple sources, with indicated Cat. No. from such selected commercial sources.

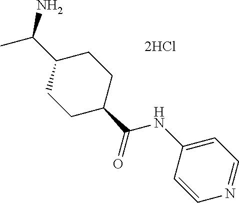 [SMILES: C[C@@H](N)[C@H]1CC[C@H](C(=O)Nc2ccncc2)CC1]

### Y-27632 (MCE, Cat. #HY-10071, CAS No.: 146986-50-7).

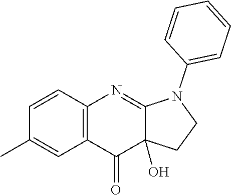 [SMILES: Cc1ccc2c(c1)C(=O)C1(O)CCN(c3ccccc3)C1=N2]

### Blebbistatin (MCE, #HY-13813, CAS No.: 674289-55-5).

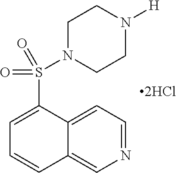 [SMILES: O=S(=O)(c1cccc2cnccc12)N1CCNCC1]

HA-100 (hydrochloride) (absin Cat. #abs47045575).

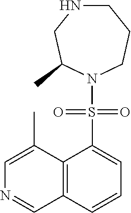 [SMILES: Cc1cncc2cccc(S(=O)(=O)N3CCCNC[C@@H]3C)c12]

### H-1152 (MCE Cat. #HY-15720, CAS No.: 451462-58-1).

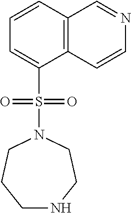 [SMILES: O=S(=O)(c1cccc2cnccc12)N1CCCNCC1]

### HA-1077 (Fasudil/AT877) (MCE Cat. #HY-10341A, CAS No.: 103745-39-7).

In some embodiments, the ROCK inhibitor is Blebbistatin. In some embodiments, the concentration of Blebbistatin is 10 μM. In some embodiments, the ROCK inhibitor is Y-27632.

In some embodiments, the duration of the step for EB formation is about 8h to about 24h, preferably about 10h to about 16h.

Differentiation of EBs into imDAP Cells

### Multistep WNT Signaling Activation

EBs are differentiated into imDAP cells through a multistep WNT signaling activation process. In some embodiments, the WNT signaling activation process comprises four substeps: step (i), step (ii), step (iii) and step (iv), also referred to herein as, S2-1, S2-2, S2-3, and S2-4, respectively. In some embodiments, the entire duration of the step for differentiation of EBs into imDAP cells is about 9 to about 20 days, preferably about 12 to about 16 days.

More specifically, the WNT signaling activation process comprises contacting the EBs with a first differentiation medium comprising a WNT signaling pathway activator at a first concentration (step (i)), contacting the cells from step (i) with a second differentiation medium comprising the WNT signaling pathway activator at a second concentration (step (ii)), contacting the cells from step (ii) with a third differentiation medium comprising the WNT signaling pathway activator at a third concentration (step (iii)), and contacting the cells from step (iii) with a fourth differentiation medium comprising the WNT signaling pathway activator at a fourth concentration (step (iv)), wherein the first and third concentrations are lower than the fourth concentration, and the second concentration is higher than the fourth concentration. The first to fourth concentrations of the WNT signaling pathway activator in the four substeps in the WNT signaling activation process disclosed herein can also be simply designated as low-high-low-intermediate. It has been demonstrated in this disclosure that the multistep WNT signaling activation process disclosed herein has led to more efficient and consistent generation of imDAP cells, as reflected by a higher percentage of FOXA2+ imDAP cells and improved batch-to-batch consistency as compared to the 2-step WNT signaling activation process and 3-step WNT signaling activation process.

In some embodiments, the first concentration of the WNT signaling pathway activator is in the range from about 0.2 μM to about 1.6 μM, preferably about 0.4 μM to about 1.6 μM, and more preferably about 0.8 μM to about 1.6 μM.

In some embodiments, the second concentration of the WNT signaling pathway activator is in the range from about 4 μM to about 10 μM, preferably about 5 μM to about 7.5 μM.

In some embodiments, the third concentration of the WNT signaling pathway activator is in the range from about 0.2 μM to about 1.6 μM, preferably about 0.4 μM to about 1.6 μM, and more preferably about 0.8 μM to about 1.6 μM. In some embodiments, the third concentration may be same as or similar to the first concentration. In some embodiments, the third concentration may be different from the first concentration.

In some embodiments, the fourth concentration of the WNT signaling pathway activator is in the range from about 2 μM to about 3.5 μM, preferably about 2 μM to about 3 μM.

### WNT Signaling Pathway Activators

The Wnt signaling pathway is an evolutionarily conserved pathway that regulates various aspects of cell fate determination, cell migration, neural patterning and organogenesis during embryonic development. See review, e.g., by Komiya and Habas (Organogenesis 2008; 4 (2): 68-75). The Wnts comprise a large family of secreted glycoproteins that bind to an Fz receptor to trigger signal transduction. The Wnt signaling pathway can be a canonical or Wnt/β-catenin dependent pathway, or a non-canonical or β-catenin-independent pathway which can be further divided into the Planar Cell Polarity and the Wnt/Ca²⁺ pathways.

The Wnt signaling pathway activators refers to agonists of the Wnt signaling pathway (e.g., agents capable of upregulating activity and/or amount of a component participating in the Wnt signaling pathway).

Non-limiting examples of Wnt signaling pathway activators include one or more of the following: a polypeptide comprising an amino acid sequence of a Wnt polypeptide, a polypeptide comprising an amino acid sequence of an activated Wnt receptor, a small organic molecule that promotes Wnt/β-catenin signaling, a small organic molecule that inhibits the expression or activity of a Wnt antagonist, an antibody that binds to and inhibits the activity of a Wnt antagonist, a polypeptide comprising an amino acid sequence of a β-catenin polypeptide, and a polypeptide comprising an amino acid sequence of a Lef-1 polypeptide, and preferably a small organic molecule that promotes Wnt/β-catenin signaling and a small organic molecule that inhibits the expression or activity of a Wnt antagonist.

Wnt signaling pathway activators can also include GSK3 inhibitors. GSK3 inhibitors may include, for example and without limitation, polynucleotides, polypeptides, and small molecules. Exemplary GSK3 inhibitors include, for example and without limitation, Kenpaullone, 1-Azakenpaullone, CHIR99021, CHIR98014, NP031112, TWS119, TWS119 pyrrolopyrimidine compound, AZD2858, AZD1080, SB415286, LY2090314, AR-A014418, CT 20026, SB216763, TDZD-8 (4-Benzyl-2-methyl-1,2,4-thiadiazolidine-3,5-dione), BIO, BIO-Acetoxime, (5-Methyl-1H-pyrazol-3-yl)-(2-phenylquinazolin-4-yl)amine, Pyridocarbazole-cyclopenadienylruthenium complex, 2-Thio(3-iodobenzyl)-5-(1-pyridyl) [1,3,4]-oxadiazole, alpha-4-Dibromoacetophenone, 3-(1-(3-Hydroxypropyl)-1H-pyrrolo[2,3-b]pyridin-3-yl]-4-pyrazin-2-yl-pyrrole-2,5-dione, L803 H-KEAPPAPPQSpP-NH2 or its myristoylated form, 2-Chloro-1-(4,5-dibromo-thiophen-2-yl)-ethanone, GF109203X, RO318220, TIBPO, and OTDZT, preferably Kenpaullone, 1-Azakenpaullone, CHIR99021, CHIR98014, NP031112, TWS119, AZD2858, AZD1080, SB415286, LY2090314, AR-A014418, SB216763, BIO-Acetoxime, (5-Methyl-1H-pyrazol-3-yl)-(2-phenylquinazolin-4-yl)amine, 2-Thio(3-iodobenzyl)-5-(1-pyridyl) [1,3,4]-oxadiazole, alpha-4-Dibromoacetophenone, 3-(1-(3-Hydroxypropyl)-1H-pyrrolo[2,3-b]pyridin-3-yl]-4-pyrazin-2-yl-pyrrole-2,5-dione, 2-Chloro-1-(4,5-dibromo-thiophen-2-yl)-ethanone, and GF109203X.

In preferred embodiments, the Wnt signaling pathway activator suitable for use herein is selected from Kenpaullone, 1-Azakenpaullone, CHIR99021, CHIR98014, NP031112, TWS119, AZD2858, AZD1080, SB415286, LY2090314, AR-A014418, CT 20026, SB216763, TDZD-8 (4-Benzyl-2-methyl-1,2,4-thiadiazolidine-3,5-dione), BIO, BIO-Acetoxime, (5-Methyl-1H-pyrazol-3-yl)-(2-phenylquinazolin-4-yl)amine, Pyridocarbazole-cyclopenadienylruthenium complex, 2-Thio(3-iodobenzyl)-5-(1-pyridyl) [1,3,4]-oxadiazole, alpha-4-Dibromoacetophenone, 3-(1-(3-Hydroxypropyl)-1H-pyrrolo[2,3-b]pyridin-3-yl]-4-pyrazin-2-yl-pyrrole-2,5-dione, TWS1 19 pyrrolopyrimidine compound, L803 H-KEAPPAPPQSpP-NH2 or its myristoylated form, 2-Chloro-1-(4,5-dibromo-thiophen-2-yl)-ethanone, GF109203X, RO318220, TIBPO, or OTDZT. In a most preferred embodiment, the Wnt signaling pathway activator is CHIR99021.

In some embodiments, the WNT signaling pathway activators in the first to fourth differentiation media are identical. In some embodiments, the WNT signaling pathway activators in the first to fourth differentiation media are different from each other. In some embodiments, the WNT signaling pathway activators in the first to fourth differentiation media comprise a glycogen synthase kinase-3 (GSK3) inhibitor. In some embodiments, the glycogen synthase kinase-3 (GSK3) inhibitor is CHIR99021.

Representative structures of certain Wnt signaling pathway activators that may be used in the culture medium of the present disclosure are provided below, many of which are widely commercially available with indicated Cat. No. from such selected commercial sources.

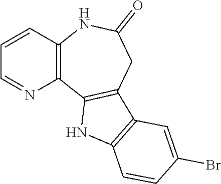 [SMILES: O=C1Cc2c([nH]c3ccc(Br)cc23)-c2ncccc2N1]

### Azakenpaullone: MCE, #HY-59090; APExBio, #B3690; Sigma-Aldrich, #A3734; CAS No.: 676586-65-9.

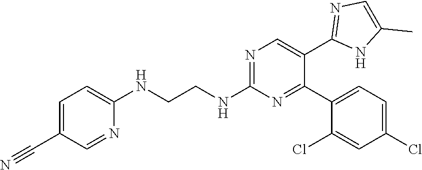 [SMILES: Cc1cnc(-c2cnc(NCCNc3ccc(C#N)cn3)nc2-c2ccc(Cl)cc2Cl)[nH]1]

### CHIR99021: APExBio, #A3011.

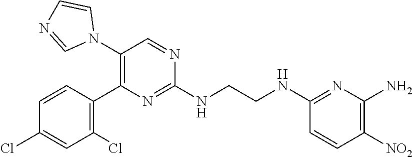 [SMILES: Nc1nc(NCCNc2ncc(-n3ccnc3)c(-c3ccc(Cl)cc3Cl)n2)ccc1[N+](=O)[O-]]

### CHIR98014: Sigma-Aldrich, #SML1094; CAS No.: 252935-94-7.

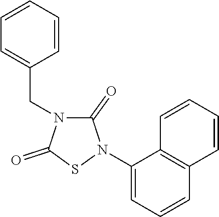 [SMILES: O=c1sn(-c2cccc3ccccc23)c(=O)n1Cc1ccccc1]

### NP031112 (Tideglusib): Sigma-Aldrich, #SML0339; APExBio, #B1539; MCE, #HY-14872; CAS No.: 865854 May 3.

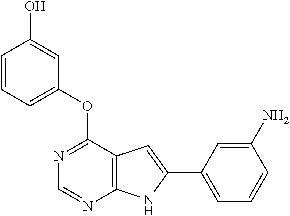 [SMILES: Nc1cccc(-c2cc3c(Oc4cccc(O)c4)ncnc3[nH]2)c1]

### TWS119: Sigma-Aldrich, #SML1271; APExBio, #B1540; MCE, #HY-10590; CAS No.: 601514-19-6.

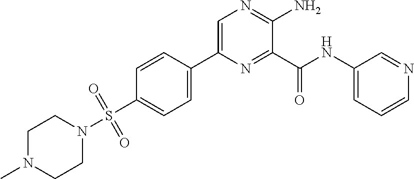 [SMILES: CN1CCN(S(=O)(=O)c2ccc(-c3cnc(N)c(C(=O)Nc4cccnc4)n3)cc2)CC1]

### AZD2858: APExBio, #B1537; MCE, #HY-15761; CAS No.: 486424-20-8.

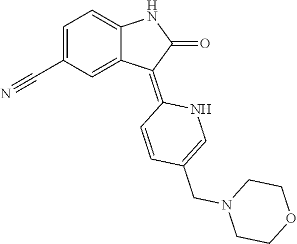 [SMILES: N#Cc1ccc2c(c1)/C(=C1\C=CC(CN3CCOCC3)=CN1)C(=O)N2]

### AZD1080: APExBio, #B1536; MCE, #HY-13862, CAS No.: 612487-72-6.

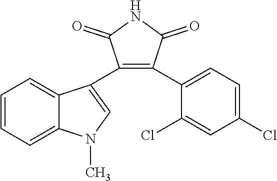 [SMILES: Cn1cc(C2=C(c3ccc(Cl)cc3Cl)C(=O)NC2=O)c2ccccc21]

### SB415286: Sigma-Aldrich, #S3567; APExBio, #A8241; MCE, #HY-15438; CAS No.: 280744-09-4.

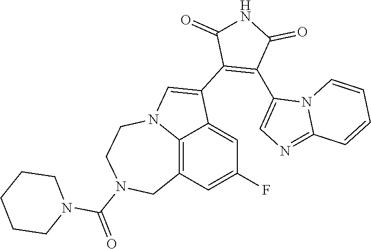 [SMILES: O=C1NC(=O)C(c2cnc3ccccn23)=C1c1cn2c3c(cc(F)cc13)CN(C(=O)N1CCCCC1)CC2]

### LY2090314: Sigma-Aldrich, #SML1438; APExBio, #A3570; MCE, #HY-16294; CAS No.: 603288-22-8.

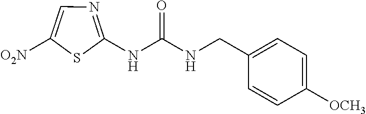 [SMILES: COc1ccc(CNC(=O)Nc2ncc([N+](=O)[O-])s2)cc1]

### AR-A014418: Sigma-Aldrich, #A3230; APExBio, #A3184; MCE, #HY-10512; CAS No.: 487021-53-3.

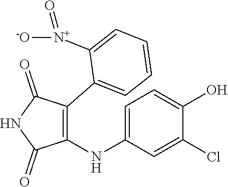

### SB216763: Sigma-Aldrich, #S3442; APExBio, #A8240; MCE, #HY-12012; CAS No.: 280744-09-4.

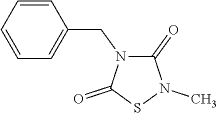

### TDZD-8: Sigma-Aldrich, #T8325; APExBio, #B1249; MCE, #HY-11012; CAS No.: 327036-89-5.

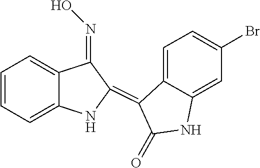 [SMILES: O=C1Nc2cc(Br)ccc2/C1=C1/Nc2ccccc2/C1=N\O]

### BIO (GSK 3 Inhibitor IX): Sigma-Aldrich, #B1686; APExBio, #B1538; MCE, #HY-10580; CAS No.: 667463-62-9.

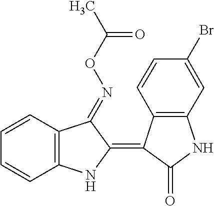 [SMILES: CC(=O)O/N=C1/C(=C2/C(=O)Nc3cc(Br)ccc32)Nc2ccccc21]

### BIO-Acetoxime; Sigma-Aldrich, #SML0531; APExBio, #B5488; MCE, #HY-15356; CAS No.: 667463-85-6.

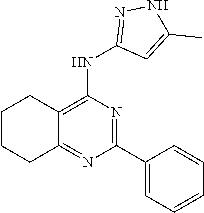 [SMILES: Cc1cc(Nc2nc(-c3ccccc3)nc3c2CCCC3)n[nH]1]

(5-Methyl-1H-pyrazol-3-yl)-(2-phenylquinazolin-4-yl)amine (GSK-3 Inhibitor XIII): MCE, #HY-112392; absin, #abs819580; aladdin, #G338805; CAS No.: 404828-08-6.

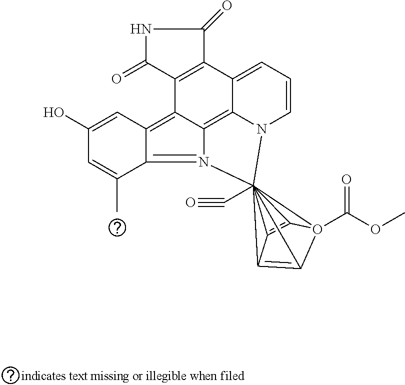

Pyridocarbazole-cyclopenadienylruthenium complex (GSK-3 Inhibitor XV): Sigma-Aldrich, #361558, CAS No.: 936112-69-5.

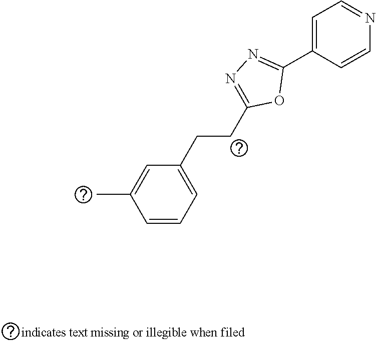 [SMILES: Cc1cccc(CCc2nnc(-c3ccncc3)o2)c1]

2-Thio(3-iodobenzyl)-5-(1-pyridyl) [1,3,4]-oxadiazole (GSK3 Inhibitor II): APExBio, #C4599; CAS No.: 478482-75-6.

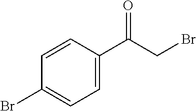 [SMILES: O=C(CBr)c1ccc(Br)cc1]

alpha-4-Dibromoacetophenone (2,4′-Dibromoacetophenone/4′-Bromophenacyl bromide): Sigma-Aldrich, #D38308; CAS No.: 99-73-0.

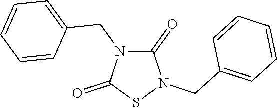 [SMILES: O=c1sn(Cc2ccccc2)c(=O)n1Cc1ccccc1]

OTDZT (2,4-dibenzyl-5-oxo thiadiazolidine-3-thione): CAS No.: 373357-10-9.

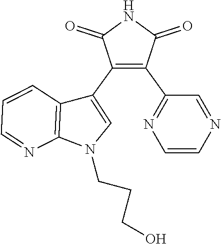 [SMILES: O=C1NC(=O)C(c2cn(CCCO)c3ncccc23)=C1c1cnccn1]

3-(1-(3-Hydroxypropyl)-1H-pyrrolo[2,3-b]pyridin-3-yl]-4-pyrazin-2-yl-pyrrole-2,5-dione (GSK-3β inhibitor XI): Sigma-Aldrich, #361553; aladdin, #G338716-1 mg; CAS No.: 626604-39-5.

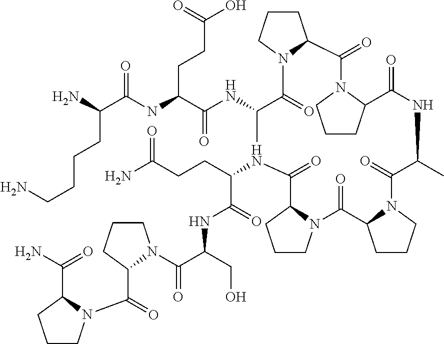

### L803 H-KEAPPAPPQSPP-NH2: CAS No.: 348089-28-1.

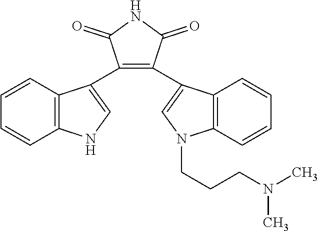 [SMILES: CN(C)CCCn1cc(C2=C(c3c[nH]c4ccccc34)C(=O)NC2=O)c2ccccc21]

### GF109203X (Bisindolylmaleimide I): Sigma-Aldrich, #G2911; APExBio, #A8342; MCE, #HY-13867; CAS No.: 133052-90-1.

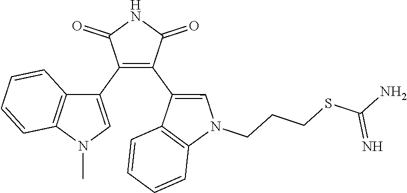 [SMILES: Cn1cc(C2=C(c3cn(CCCSC(=N)N)c4ccccc34)C(=O)NC2=O)c2ccccc21]

### RO318220: MCE, #HY-13866A; CAS No.:125314-64-9.

In certain embodiments, the differentiation medium(s) of the present disclosure further comprise(s) a BMP4 inhibitor, a TGF-β inhibitor, and a sonic hedgehog agonist.

### Inhibition of BMP and TGF-β Signaling

In the multistep WNT signaling activation process, the WNT signaling activation may be combined with a dual inhibition of a SMAD signaling, that is, BMP and TGF-β signaling. In certain embodiments, the combination of the WNT signaling activation with the dual inhibition of BMP and TGF-β signaling can further improve the specification of imDAP cells.

### BMP4 Inhibitors

The BMP family inhibitor is a substance that inhibits BMP signaling through binding between BMP (bone morphogenetic protein) and a BMP receptor (type I or type II). A BMP inhibitor suitable for use herein includes proteinaceous inhibitors and small molecule inhibitors. Examples of such proteinaceous inhibitors include Noggin, chordin and follostatin. Examples of such small molecule inhibitors include Dorsomorphin and its derivatives, LDN193189 and its derivatives. These compounds are commercially available and are readily available. Preferably, LDN193189 is used. In certain embodiments, the BMP4 inhibitor comprises Dorsomorphin, LDN193189 or a combination thereof.

Representative structures of certain BMP4 inhibitors that may be used in the culture medium of the present disclosure are provided below, many of which are widely commercially available with indicated Cat. No. from such selected commercial sources.

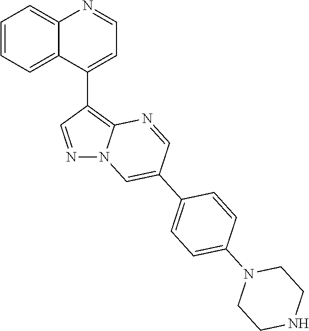 [SMILES: c1ccc2c(-c3cnn4cc(-c5ccc(N6CCNCC6)cc5)cnc34)ccnc2c1]

### LDN193189 (MCE Cat. #HY-12071A, CAS No.: 1062368-24-4).

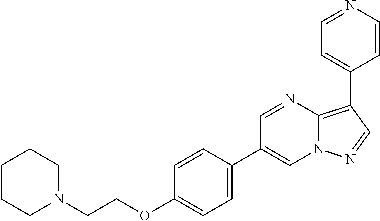 [SMILES: c1cc(-c2cnn3cc(-c4ccc(OCCN5CCCCC5)cc4)cnc23)ccn1]

### Dorsomorphin (Sigma-Aldrich Cat. #P5499; APExBio Cat. #B3252; MCE Cat. #HY-13418A; CAS No.: 866405-64-3).

### TGF-β Inhibitors

TGF-β signaling typically begins with binding of a TGF-β superfamily ligand to a Type II receptor, which recruits and phosphorylates a Type I receptor. The Type I receptor then phosphorylates SMADs, which act as transcription factors in the nucleus and regulate target gene expression. Alternatively, TGF-β signaling can activate MAP kinase signaling pathways, for example, via p38 MAP kinase.

The TGF-β inhibitor as used herein include an agent that reduces the activity of the TGF-β signaling pathway. There are many different ways of disrupting the TGF-β signaling pathway. For example, TGF-β signaling may be disrupted by: inhibition of TGF-β expression by a small-interfering RNA strategy; inhibition of furin (a TGF-β activating protease); inhibition of the pathway by physiological inhibitors, such as inhibition of BMP by Noggin, DAN or DAN-like proteins; neutralization of TGF-β with a monoclonal antibody; inhibition with small-molecule inhibitors of TGF-β receptor kinase 1 (also known as activin receptor-like kinase, ALK5), ALK4, ALK6, ALK7 or other TGF-β-related receptor kinases; inhibition of Smad 2 and Smad 3 signaling by overexpression of their physiological inhibitor, Smad 7, or by using thioredoxin as an Smad anchor disabling Smad from activation.

For example, a TGF-β inhibitor may target a serine/threonine protein kinase selected from: TGF-β receptor kinase 1, ALK4, ALK5, ALK7, or p38. ALK4, ALK5 and ALK7 are all closely related receptors of the TGF-β superfamily. An inhibitor of any one of these kinases is one that effects a reduction in the enzymatic activity of any one (or more) of these kinases.

In certain embodiments, a TGF-β inhibitor may bind to and inhibit the activity of a Smad protein, such as R-SMAD or SMAD1-5 (i.e., SMAD1, SMAD2, SMAD3, SMAD4 or SMAD5).

In certain embodiments, a TGF-β inhibitor may bind to and reduces the activity of Ser/Thr protein kinase selected from: TGF-β receptor kinase 1, ALK4, ALK5, ALK7, or p38.

A TGF-β inhibitor suitable for use herein may be a protein, a peptide, a small-molecule, a small-interfering RNA, an antisense oligonucleotide, an aptamer, an antibody or an antigen-binding portion thereof. The inhibitor may be naturally occurring or synthetic. Examples of small-molecule TGF-β inhibitors that can be used in the context of the present disclosure include, but are not limited to, RepSox (2-[5-(6-methylpyridin-2-yl)-1H-pyrazol-4-yl]-1,5-naphthyridine), SB431542, SB505124, LY36494, SJN-2511, A83-01, D4476, GW788388, LY364947, LY580276, SB525334, SD208, GW6604, and any combination thereof. Preferably, SB431542 is used.

In certain embodiments, the TGF-β inhibitor is selected from the group consisting of RepSox, A83-01, SB431542, D4476, GW788388, LY364947, SB525334, SB505124, SD208, GW6604, and any combination thereof.

Representative structures of certain TGF-β inhibitors suitable for use herein are provided below, many of which are widely commercially available from multiple sources, with indicated Cat. No. from such selected commercial sources.

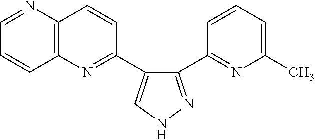 [SMILES: Cc1cccc(-c2n[nH]cc2-c2ccc3ncccc3n2)n1]

RepSox (2-[5-(6-methylpyridin-2-yl)-1H-pyrazol-4-yl]-1,5-naphthyridine) (Sigma-Aldrich Cat. #R0158; APExBio Cat. #A3754; MCE Cat. #HY-13012; CAS No.: 446859-33-2).

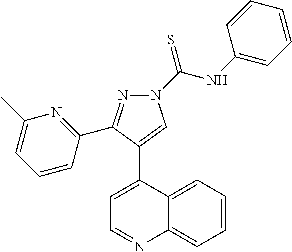 [SMILES: Cc1cccc(-c2nn(C(=S)Nc3ccccc3)cc2-c2ccnc3ccccc23)n1]

### A83-01 (Sigma-Aldrich Cat. #SML0788; APExBio Cat. #A3133; MCE Cat. #HY-10432; CAS No.: 909910-43-6).

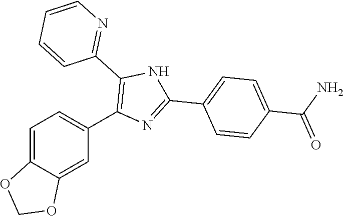 [SMILES: NC(=O)c1ccc(-c2nc(-c3ccc4c(c3)OCO4)c(-c3ccccn3)[nH]2)cc1]

### SB431542 (APExBio Cat. #A8249, CAS No.: 301836-41-9).

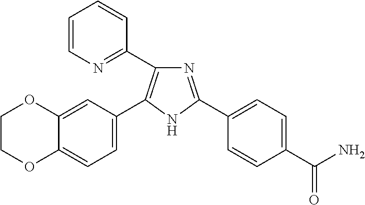 [SMILES: NC(=O)c1ccc(-c2nc(-c3ccccn3)c(-c3ccc4c(c3)OCCO4)[nH]2)cc1]

### D4476 (Sigma-Aldrich Cat. #D1944; APExBio Cat. #A3342; MCE Cat. #HY-10324; CAS No.: 301836-43-1).

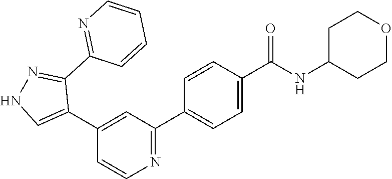 [SMILES: O=C(NC1CCOCC1)c1ccc(-c2cc(-c3c[nH]nc3-c3ccccn3)ccn2)cc1]

### GW788388 (APExBio Cat. #A8301; MCE Cat. #HY-10326; CAS No.: 452342-67-5).

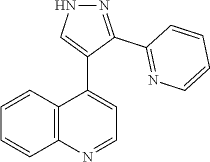 [SMILES: c1ccc(-c2n[nH]cc2-c2ccnc3ccccc23)nc1]

### LY364947 (Sigma-Aldrich Cat. #L6293; APExBio Cat. #B2287; MCE Cat. #HY-31462; CAS No.: 396129-53-6).

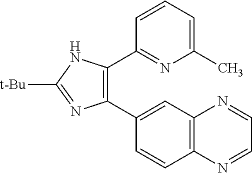 [SMILES: Cc1cccc(-c2[nH]c(C(C)(C)C)nc2-c2ccc3nccnc3c2)n1]

### SB525334 (Sigma-Aldrich Cat. #S8822; APExBio Cat. #A5602; MCE Cat. #HY-12043; CAS No.: 356559-20-1).

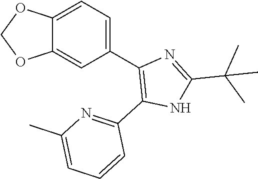 [SMILES: Cc1cccc(-c2[nH]c(C(C)(C)C)nc2-c2ccc3c(c2)OCO3)n1]

### SB505124 (APExBio Cat. #B2289; MCE Cat. #HY-13521; CAS No.: 694433-59-5).

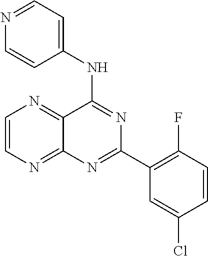 [SMILES: Fc1ccc(Cl)cc1-c1nc(Nc2ccncc2)c2nccnc2n1]

### SD208 (Sigma-Aldrich Cat. #S7071; APExBio Cat. #A3808; MCE Cat. #HY-10324; CAS No.: 627536-09-8).

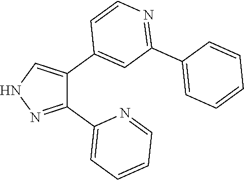 [SMILES: c1ccc(-c2cc(-c3c[nH]nc3-c3ccccn3)ccn2)cc1]

GW6604 (absin Cat. #abs814099; CAS No.: 452342-37-9).

In some embodiments, the first differentiation medium further comprises a BMP4 inhibitor, a TGF-β inhibitor, and a sonic hedgehog agonist. In some embodiments, the second to fourth differentiation media each further comprise none, one or more of a BMP4 inhibitor, a TGF-β inhibitor, or a sonic hedgehog agonist. In some embodiments, a BMP4 inhibitor is present in each of the first to fourth differentiation media. In some embodiments, a BMP4 inhibitor is present in the first differentiation medium, but not in the second, third and/or fourth differentiation media. In some embodiments, a TGF-β inhibitor is present in the first and second differentiation media, but not in the third and/or fourth differentiation media. In some embodiments, a TGF-β inhibitor is present in the first differentiation medium, but not in the second, third and fourth differentiation media. In some embodiments, a sonic hedgehog agonist is present in the first, second and third differentiation media, but not in the fourth differentiation medium. In some embodiments, the first and second differentiation media comprise a BMP4 inhibitor, a TGF-β inhibitor, and a sonic hedgehog agonist, the third differentiation medium comprises a BMP4 inhibitor and a sonic hedgehog agonist, without a TGF-β inhibitor, and the fourth differentiation medium comprises a BMP4 inhibitor, without a TGF-β inhibitor and a sonic hedgehog agonist.

### Sonic Hedgehog (SHH) Agonists

The sonic hedgehog (SHH) agonist, also referred to as SHH signal-stimulating agent, used in the present disclosure is a substance that causes disinhibition of Smoothened (Smo) due to binding of SHH to its receptor, Patched (Ptch1), and also causes activation of Gli2, which follows the disinhibition. The SHH agonist may be of a small organic molecule and a polypeptide. Examples of SHH agonists include SHH, SHH C24II, SHH C25II, SAG, SMO-IN-1, purmorphamine, and analogues thereof.

Representative structures of certain SHH agonists suitable for use herein are provided below, many of which are widely commercially available, with indicated Cat. No. from such selected commercial sources.

SHH: Pepprotech, #100-45, with an amino acid sequence of:

(SEQ ID NO: 1)
IVIGPGRGFGKRRHPKKLTPLAYKQFIPNVAEKTLGASGRYEGKISRNSE
RFKELTPNYNPDIIFKDEENTGADRLMTQRCKDKLNALAISVMNQWPGVK
LRVTEGWDEDGHHSEESLHYEGRALDITTSDRDRSKYGMLARLAVEAGFD
WVYYESKAHIHCSVKAENSVAAKSGG

SHH C24II: Nuwacell; Procell, #PCK216; MCE, #HY-P7407, with an amino acid sequence of:

(SEQ ID NO: 2)
IIGPGRGFGKRRHPKKLTPLAYKQFIPNVAEKTLGASGRYEGKISRNSER
FKELTPNYNPDIIFKDEENTGADRLMTQRCKDKLNALAISVMNQWPGVKL
RVTEGWDEDGHHSEESLHYEGRAVDITTSDRDRSKYGMLARLAVEAGFDW
VYYESKAHIHCSVKAENSVAAKSGG

SHH C25II: Nuwacell; Procell, #PCK216; MCE, #HY-P7407, with an amino acid sequence of:

(SEQ ID NO: 3)
IIGPGRGFGKRRHPKKLTPLAYKQFIPNVAEKTLGASGRYEGKITRNSER
FKELTPNYNPDIIFKDEENTGADRLMTQRCKDKLNALAISVMNQWPGVKL
RVTEGWDEDGHHSEESLHYEGRAVDITTSDRDRSKYGMLARLAVEAGFDW
VYYESKAHIHCSVKAENSVAAKSGG

Purmorphamine: CAS No.: 483367-10-8

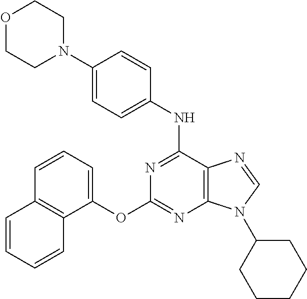 [SMILES: c1ccc2c(Oc3nc(Nc4ccc(N5CCOCC5)cc4)c4ncn(C5CCCCC5)c4n3)cccc2c1]

SAG: APExBio, #B5837, CAS No.: 912545-86-9

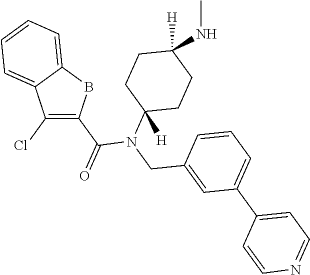

SMO-IN-1: MCE, #HY-147743, CAS No.: 1126365-66-9

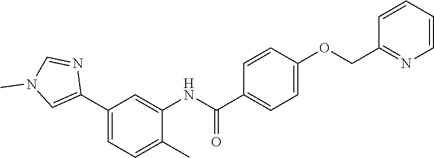 [SMILES: Cc1ccc(-c2cn(C)cn2)cc1NC(=O)c1ccc(OCc2ccccn2)cc1]

In some embodiments, the concentration of the BMP4 inhibitor, if present in the first, second, third or fourth differentiation medium, is in the range from about 0.05 μM to about 2.0 μM, preferably about 0.1 μM to about 1.0 μM. The optimum concentration of BMP family inhibitor can be set through preliminary experiments.

In some embodiments, the concentration of the TGF-β inhibitor, if present in the first, second, third or fourth differentiation medium, is in the range from about 1 μM to about 100 μM, preferably about 2.0 μM to about 50 μM, for example, 10.0 μM.

In some embodiments, the concentration of the sonic hedgehog agonist, if present in the first, second, third or fourth differentiation medium, is in the range from about 100 ng/ml to about 2000 ng/mL, preferably about 300 ng/mL to about 500 ng/ml.

In some embodiments, the first differentiation medium comprises CHIR99021, LDN193189, SB431542, and SHH C24II, and the second to fourth differentiation media each comprise CHIR99021 and none, one or more of LDN193189, SB431542, and SHH C24II. For example, the first differentiation medium comprises CHIR99021, LDN193189, SB431542, and SHH C24II, the second differentiation medium comprises CHIR99021, LDN193189, SB431542, and SHH C24II, the third differentiation medium comprises CHIR99021, LDN193189, and SHH C24II, without SB431542, and the fourth differentiation medium comprises CHIR99021 and LDN193189, without SB431542 and/or SHH C24II. In some embodiments, the first differentiation medium comprises CHIR99021, LDN193189, SB431542, SHH C24II, Purmorphamine and SAG; and the second to fourth differentiation media each comprises CHIR99021, and none, one or more of LDN193189, SB431542, SHH C24II, Purmorphamine, and SAG. For example, the first differentiation medium comprises CHIR99021, LDN193189, SB431542, SHH C24II, Purmorphamine and SAG, the second differentiation medium comprises CHIR99021, LDN193189, SB431542, SHH C24II, Purmorphamine, and SAG, the third differentiation medium comprises CHIR99021, LDN193189, SHH C24II, Purmorphamine, and SAG, without SB431542, and the fourth differentiation medium comprises CHIR99021 and LDN193189, without SB431542, SHH C24II, Purmorphamine, and SAG.

### Fibroblast Growth Factors

In certain embodiments, the differentiation medium of the present disclosure further comprises a fibroblast growth factor (FGF). In certain embodiments, the fibroblast growth factor comprises FGF2 (e.g., Nuwacell), FGF1 (e.g., MCE Cat. #HY-P7001), FGF8 (e.g., MCE Cat. #HY-P7347, MCE Cat. #HY-P7349, MCE Cat. #HY-P7350) and/or FGF20 (e.g., R&D Cat. #2547-FG).

In the present disclosure, the FGF8 is not limited, and in case of human FGF8, examples of the FGF8 include the following four splicing forms: FGF8a, FGF8b, FGF8e, and FGF8f. The FGF8 in the present disclosure is more preferably FGF8b.

In some embodiments, the concentration of fibroblast growth factor (e.g., FGF8) can be, for example, about 10 ng/ml to about 1000 ng/ml, preferably about 50 ng/ml to about 300 ng/ml. The optimal concentration of FGF can be determined through routine experiments.

In some embodiments, the first to fourth differentiation media further comprise a neural basal medium. In some embodiments, the first to fourth differentiation media further comprise a serum-free and xeno-free neural basal medium. The neural basal medium can be produced by adding one or more neural growth supplements into one or more common basal media. Examples of the common basal medium comprises DMEM: F12 (e.g., Gibco Cat. #C11330500BT), BME medium (e.g., Gibco Cat. #21010046, or Sigma-Aldrich Cat. #B9638), IMDM medium (e.g., Gibco Cat. #12440053; or Sigma-Aldrich Cat. #13390), Eagle MEM medium (e.g., Minimum Essential Medium (MEM), developed by Harry Eagle, Sigma-Aldrich Cat. #M2414/M2279/M5690), α-MEM medium (e.g., Gibco Cat. #12561056; or, Sigma-Aldrich Cat. #M0894), DMEM medium (e.g., Gibco Cat. #21068028), RPMI 1640 medium (e.g., Gibco Cat. #11875093), Ham's F12 medium (e.g., Gibco Cat. #11765054), or a mixture thereof. Other examples of the basal medium comprises NEUROBASAL™ basal medium (e.g., Gibco Cat. #21103049), NEUROBASAL-ATM basal medium (e.g., Gibco Cat. #10888022), NEUROBASAL PLUS™ basal medium (e.g., Gibco Cat. #A3582901), and/or BRAINPHYS™ basal medium (e.g., STEMCELL Cat. #05790). In some embodiments, the neural growth supplement is selected from the group consisting of B27, N1, N2, and any combination thereof. In some embodiments, the neural growth supplement comprises B27 (e.g., B27 from GIBCO BRL Cat. #12587010).

Examples of the neural basal medium comprise DMEM: F12/B27, DMEM: F12/N2, DMEM: F12/N1, Neurobasal/B27, Neurobasal/N2, Neurobasal/N1, DMEM: F12+Neurobasal/B27, DMEM: F12+Neurobasal/N2, DMEM: F12+Neurobasal/N1, IMDM/B27, IMDM/N2, IMDM/N1, RPMI 1640/B27, RPMI 1640/N2, or RPMI 1640/N1.

In some embodiments, the neural growth supplement is present in the culture medium at a concentration of about 0.1% to about 20% by volume, preferably about 0.1% to about 10% by volume, and more preferably about 0.5% to about 5% by volume.

### Ascorbic Compound

It has been uniquely discovered in accordance with the present disclosure that addition of ascorbic compound to the neural basal medium leads to higher efficiency for differentiation, as reflected by higher percentage of FOXA2+ cells. Accordingly, in some embodiments, the neural basal medium comprises an ascorbic compound. Non-limiting examples of the ascorbic compound comprises ascorbic acid (e.g., L-ascorbic acid), dehydrogenated ascorbic acid, or their salts, analogues or derivatives. In some embodiments, the neural basal medium comprises ascorbic acid. In some embodiments, the neural basal medium comprises L-ascorbic acid 2-phosphate sesquimagnesium salt.

In some embodiments, the concentrations of the ascorbic compound in the first to fourth differentiation media each are about 5 μg/mL to about 100 μg/mL, preferably about 10 μg/mL to about 50 μg/mL.

### Vitamin A

In some embodiments, the neural basal medium in each of the first to fourth differentiation media contains no vitamin A. In some embodiments, vitamin A is added to the neural basal medium during the initial days of differentiation. In some embodiments, the neural basal medium in the first differentiation medium contains vitamin A and the neural basal medium in the second to fourth differentiation media contains no vitamin A. In case of containing vitamin A in a neural basal medium, it can be provided by a neural growth supplement such as B27 (plus vitamin A), or can be externally added into the neural basal medium.

In some embodiments, the neural basal medium in each of the first to fourth differentiation media is free of vitamin A. It has been further discovered in accordance with the present disclosure that omission of vitamin A in each of the first to fourth differentiation media leads to higher efficiency for differentiation. In some embodiments, a neural basal medium comprising an ascorbic compound and free of vitamin A is used, which results in the optimal efficiency for differentiation.

Further, it has been demonstrated herein that the time window (timing) during which the cells are exposed to the second (high, or “boost”) concentration of the WNT signaling pathway activator (i.e., step (ii)) can impact the efficiency of imDAP differentiation. In some embodiments, the cells are exposed to the second (high, or “boost”) concentration of the WNT signaling pathway activator from Day 3 to Day 5. In some embodiments, the cells are exposed to the second concentration of the WNT signaling pathway activator from Day 3 to Day 6. In some embodiments, the cells are exposed to the second concentration of the WNT signaling pathway activator from Day 3 to Day 7. In some embodiments, the cells are exposed to the second concentration of the WNT signaling pathway activator from Day 3 to Day 8. In some embodiments, the cells are exposed to the second (high, or “boost”) concentration of the WNT signaling pathway activator from Day 4 to Day 6. In some embodiments, the cells are exposed to the second concentration of the WNT signaling pathway activator from Day 4 to Day 7. In some embodiments, the cells are exposed to the second concentration of the WNT signaling pathway activator from Day 4 to Day 8. In some embodiments, the cells are exposed to the second (high, or “boost”) concentration of the WNT signaling pathway activator from Day 5 to Day 7. In some embodiments, the cells are exposed to the second concentration of the WNT signaling pathway activator from Day 5 to Day 8. In accordance with this disclosure, the time window of from Day 4 to Day 6, from Day 4 to Day 7, or from Day 4 to Day 8 for step (ii) and in particular from Day 4 to Day 8 can result in a higher percentage of imDAP cells positive for OTX2, for example, as compared to other time windows. For purpose of clarification, as used herein, Day 0 represents the time point when the step (ii) starts or the differentiation is initiated.

It has been demonstrated herein that the time window (timing) during which the cells are exposed to the BMP inhibitor, and the time window during which the cells are exposed to the TGF-β inhibitor and/or the concentrations of the inhibitors during exposure can impact the efficiency of imDAP differentiation. In some embodiments, the timing for treatment with the BMP inhibitor (e.g., LDN193189) in the multistep WNT signaling activation process is from Day 0 to Day 4, and the timing for treatment with the TGF-β inhibitor (e.g., SB431542) is from Day 0 to Day 4. In some embodiments, the timing for treatment with the BMP inhibitor (e.g., LDN193189) in the multistep WNT signaling activation process is from Day 0 to Day 6, and the timing for treatment with the TGF-β inhibitor (e.g., SB431542) is from Day 0 to Day 4. In some embodiments, the timing for treatment with the BMP inhibitor (e.g., LDN193189) in the multistep WNT signaling activation process is from Day 0 to Day 8, and the timing for treatment with the TGF-β inhibitor (e.g., SB431542) is from Day 0 to Day 4. In some embodiments, the timing for treatment with the BMP inhibitor (e.g., LDN193189) in the multistep WNT signaling activation process is from Day 0 to Day 12, and the timing for treatment with the TGF-β inhibitor (e.g., SB431542) is from Day 0 to Day 4. In some embodiments, the timing for treatment with the BMP inhibitor (e.g., LDN193189) in the multistep WNT signaling activation process is from Day 0 to Day 4, and the timing for treatment with the TGF-β inhibitor (e.g., SB431542) is from Day 0 to Day 6. In some embodiments, the timing for treatment with the BMP inhibitor (e.g., LDN193189) in the multistep WNT signaling activation process is from Day 0 to Day 6, and the timing for treatment with the TGF-β inhibitor (e.g., SB431542) is from Day 0 to Day 6. In some embodiments, the timing for treatment with the BMP inhibitor (e.g., LDN193189) in the multistep WNT signaling activation process is from Day 0 to Day 8, and the timing for treatment with the TGF-β inhibitor (e.g., SB431542) is from Day 0 to Day 6. In some embodiments, the timing for treatment with the BMP inhibitor (e.g., LDN193189) in the multistep WNT signaling activation process is from Day 0 to Day 12, and the timing for treatment with the TGF-β inhibitor (e.g., SB431542) is from Day 0 to Day 6. In some embodiments, the timing for treatment with the BMP inhibitor (e.g., LDN193189) in the multistep WNT signaling activation process is from Day 0 to Day 4, and the timing for treatment with the TGF-β inhibitor (e.g., SB431542) is from Day 0 to Day 8. In some embodiments, the timing for treatment with the BMP inhibitor (e.g., LDN193189) in the multistep WNT signaling activation process is from Day 0 to Day 6, and the timing for treatment with the TGF-β inhibitor (e.g., SB431542) is from Day 0 to Day 8. In some embodiments, the timing for treatment with the BMP inhibitor (e.g., LDN193189) in the multistep WNT signaling activation process is from Day 0 to Day 8, and the timing for treatment with the TGF-β inhibitor (e.g., SB431542) is from Day 0 to Day 8. In some embodiments, the timing for treatment with the BMP inhibitor (e.g., LDN193189) in the multistep WNT signaling activation process is from Day 0 to Day 12, and the timing for treatment with the TGF-β inhibitor (e.g., SB431542) is from Day 0 to Day 8.

In accordance with this disclosure, suitable time windows and/or concentrations of the inhibitors can result in a cell population with higher percentage or number of imDAP cells positive for FOXA2 and OTX2 as well as LMX1A. For example, in some embodiments, the timing for treatment with the BMP inhibitor in the multistep WNT signaling activation process is from Day 0 to Day 6, and the timing for treatment with the TGF-β inhibitor is from Day 0 to Day 4. For example, in some embodiments, the concentration of the BMP inhibitor is 0.5 μM, and the concentration of the TGF-β inhibitor is 20 μM. For example, in some embodiments, the concentration of the BMP inhibitor is 0.1 μM, and the concentration of the TGF-β inhibitor is 50 μM. For example, in some embodiments, the concentration of the BMP inhibitor is 0.2 μM, the concentration of the TGF-β inhibitor is 50 μM, the timing for treatment of the BMP inhibitor in the multistep WNT signaling activation process is from Day 0 to Day 6, and the timing for treatment of the TGF-β inhibitor in the multistep WNT signaling activation process is from Day 0 to Day 4, or from Day 0 to Day 6. For example, in some embodiments, the concentration of the BMP inhibitor is 0.5 μM, the concentration of the TGF-β inhibitor is 20 μM, the timing for treatment of the BMP inhibitor in the multistep WNT signaling activation process is from Day 0 to Day 6, or from Day 0 to Day 8, and the timing for treatment of the TGF-β inhibitor in the multistep WNT signaling activation process is from Day 0 to Day 4.

In some embodiments, the duration for step (i) is about 3 to about 4 days, and preferably about 4 days. In some embodiments, for substage S2-1/step (i), the EBs are cultured in a first differentiation medium comprising a WNT signaling pathway activator at a first (low) concentration that is in the range from about 0.2 μM to about 1.6 μM, preferably about 0.4 μM to about 1.6 μM, and preferably about 0.8 μM to 1.6 μM, for a duration of about 3 to about 4 days, preferably about 4 days.

In some embodiments, the duration for step (ii) is about 2 to about 5 days, and preferably about 2 to about 4 days. In some embodiments, for substage S2-2/step (ii), the cells from S2-1/step (i) are cultured in a second differentiation medium comprising a WNT signaling pathway activator (e.g., the same WNT signaling pathway activator used in S2-1/step (i)) at a second (high) concentration that is in the range from about 4 μM to about 10 μM, preferably about 5 μM to about 7.5 μM, for a duration of about 2 to about 5 days, preferably about 2 to about 4 days, e.g., about 2 days, about 3 days, or about 4 days.

In some embodiments, the duration for step (iii) is about 2 to about 5 days, and preferably about 2 days. In some embodiments, for substage S2-3/step (iii), the cells from S2-2/step (ii) are cultured in a third differentiation medium comprising a WNT signaling pathway activator (e.g., the same WNT signaling pathway activator used in a previous step or steps) at a third (low) concentration that is in the range from about 0.2 μM to about 1.6 μM, preferably about 0.4 μM to about 1.6 μM, preferably about 0.8 μM to 1.6 μM, for a duration of about 2 to about 5 days, preferably about 2 days.

In some embodiments, the duration for step (iv) is about 2 to about 6 days, and preferably about 3 to about 6 days. In some embodiments, for substage S2-4/step (iv), the cells from S2-3/step (iii) are cultured in a fourth differentiation medium comprising a WNT signaling pathway activator (e.g., the same WNT signaling pathway activator used in a previous step or steps) at a fourth (intermediate) concentration (S2-4) that is in the range from about 2 μM to about 3.5 μM, preferably about 2 μM to 3 μM, for a duration of about 2 to about 6 days, preferably about 3 to about 6 days.

Expansion and Enrichment of imDAP Cells

In the method disclosed herein, the differentiated imDAP cells or population thereof can be expanded. The imDAP cells or population thereof can be expanded in an expansion medium. In accordance with this disclosure, any well known expansion medium or any other suitable expansion medium may be used herein. In some embodiments, the expansion medium suitable for use herein can be a chemically defined serum-free expansion medium that is capable of promoting robust expansion of imDAP cells. In some embodiments, an expansion medium comprising (a) a basal medium; (b) a neural growth supplement; (c) a WNT signaling pathway activator; (d) a Rho Kinase (ROCK) inhibitor; (c) a Transforming Growth Factor β (TGF-β) inhibitor; and (f) a BMP inhibitor is used.

In some embodiments, the expansion medium comprises a base medium (e.g., DMEM: F12/B27 (99:1, v/v)) supplemented with a ROCK inhibitor (e.g., Y-27632) at e.g., 5-15 μM, a WNT signaling activator (e.g., CHIR99021) at e.g., 2-4 μM, a BMP inhibitor (e.g., LDN193189) at e.g., 0.05-0.5 μM, and a TGF-β inhibitor (e.g., SB431542) at e.g., 3-7 μM. In some embodiments, the expansion medium comprises DMEM: F12/B27 (99:1, v/v), 10 μM Y-27632, 0.1 μM LDN193189, 5 μM SB431542, and 3 μM CHIR99021.

The duration of expansion may last about 3-13 days, preferably about 5-8 days.

The method disclosed herein may comprise enriching the imDAP cells by sorting for TPBG+ cells. The term “enriching,” “enriched,” or “enrichment” refers to increase of the percentage of imDAP cells expressing specific marker(s) in the cell population. In some embodiments, the imDAP cells can be enriched by sorting via Fluorescence Activated Cell Sorting (FACS) or Magnetic Activated Cell Sorting (MACS) for cell surface markers of imDAP cells. The step for enrichment can be performed at any time point after the initiation of differentiation, for example, during differentiation, expansion and/or maturation, after differentiation, expansion and/or maturation, or before expansion and/or maturation.

TPBG, also known as trophoblast glycoprotein, 5T4, or WNT activated inhibitory factor 1 (WAIF1), is expressed in trophoblasts and several carcinomas. It has been showed that TPBG has higher expression in LMX1A+ cells relative to LMX1A-cells. Thus, TPBG is an effective candidate marker for sorting of LMX1A+ imDAP cells.

In the method disclosed herein, the timing for imDAP enrichment can impact the sorting efficiency of LMX1A+ imDAP cells. In some embodiments, the imDAP cells are enriched by sorting for TPBG+ cells during differentiation. In some embodiments, the imDAP cells are enriched by sorting for TPBG+ cells after differentiation and before expansion. In some embodiments, the imDAP cells are enriched by sorting for TPBG+ cells after expansion for one or more passages. In some embodiments, the imDAP cells are enriched by sorting for TPBG+ cells after expansion for one passage. In accordance with this disclosure, enriching the imDAP cells by sorting for TPBG+ cells after differentiation and before expansion, or enriching the imDAP cells by sorting for TPBG+ cells after expansion for one passage, and particularly the latter, can result in more efficient sorting of imDAP cells.

In the method disclosed herein, the seeding density for expansion for one passage after differentiation can impact the sorting efficiency of LMX1A+ imDAP cells. In some embodiments, the imDAP cells are expanded at a seeding density of about 1×10⁴ cells/cm² to about 8×10⁴/cm². In accordance with this disclosure, a low seeding density for expansion for one passage after differentiation can improve the efficiency and recovery for the sorting of LMX1A+ imDAP cells in the cell population after the expansion. In some embodiments, the imDAP cells are expanded at a seeding density of about 1×10⁴ cells/cm² to about 3×10⁴ cells/cm².

After differentiation, at least about 78%, preferably at least about 85%, more preferably at least 90%, and most preferably 95% to 100% of the cells in the cell population without any enrichment are FOXA2+ imDAP cells. After differentiation, at least about 60%, preferably from about 60% to about 90%, more preferably about 70% to about 90%, and most preferably 80% to 90% of the cells in the cell population without any enrichment are FOXA2+OTX2+ imDAP cells. After differentiation, at least about 50%, preferably from about 50% to about 90%, more preferably about 50% to about 80%, and most preferably 60% to 80% of the cells in the cell population without any enrichment are LMX1A+ imDAP cells.

The percentage of imDAP cells expressing specific marker(s) such as LMX1A and OTX2 in the cell population can be significantly increased by enrichment. In some embodiments, the percentage of LMX1A+ imDAP cells is increased by about 20-35% after enrichment. In some embodiments, the percentage of OTX2+ imDAP cells is increased by about 20-35% after enrichment.

In some embodiments, after enrichment, at least about 90%, preferably from about 95%, more preferably at least about 98% and most preferably 100% of the cells in the cell population obtained are LMX1A+ imDAP cells.

In some embodiments, after enrichment, at least about 90%, preferably from about 95%, more preferably at least about 98% and most preferably 100% of the cells in the cell population obtained are OTX2+ imDAP cells.

In some embodiments, the imDAP cells are FOXA2+OTX2+EN1+LMX1A+PAX6−NKX2.1− cells.

### Cryopreservation

The cells or population thereof derived from any process such as differentiation, expansion, maturation, and/or enrichment in the method disclosed herein can be cryopreserved. In some embodiments, the cells or population thereof are cryopreserved after differentiation and before expansion, maturation, and/or enrichment. In some embodiments, the cells or population thereof are cryopreserved after expansion, maturation, and/or enrichment.

Cryopreservation is a process that involves cooling cells to very low temperatures (−80° C. to −196° C.) and suspending their cellular metabolism, which preserves the cells for any considerable period. The main techniques documented in the art include controlled rate and slow freezing and a flash-freezing process known as vitrification. Controlled-rate and slow freezing, also known as slow programmable freezing (SPF), is a technique where cells are cooled to around −196° C. over the course of several hours. Vitrification is a flash-freezing (ultra-rapid cooling) process that helps to prevent the formation of ice crystals and helps prevent cryopreservation damage.

In some embodiments, imDAP cells are crypreserved by controlled-rate and slow freezing. Cells can be suspended in a freezing media. Various commercially available freezing media may be used, such as such as CryoStor® CS10 (Stem Cell Technologies) and mFreSR™ (Stem Cell Technologies). The cell suspension can be aliquoted into cryogenic vials, such as Corning® Cryogenic Vials (Catalog #100-0091, 100-0095). Controlled-rate freezing is utilized and is achieved by using a controlled-rate freezer or by placing cryogenic vials in an isopropanol freezing container (e.g., Nalgene® Mr. Frosty) or an isopropanol-free container such as Corning® CoolCell® and into a −80° C. freezer to cool slowly overnight. For long term storage, the cryogenic vials can be transferred to liquid nitrogen tanks (−135° C. to −196° C.).

### Compositions

In another aspect of the disclosure, a substantially homogenous population of imDAP cells is provided and can be prepared by the method described herein. By “substantially homogeneous population” it is meant that a substantial number of the total population of the cells are of the same or similar type and/or are in the same or similar state of differentiation. The method described herein provides a substantially homogenous population of imDAP cells wherein at least about 78%, preferably at least about 85%, more preferably at least 90%, and most preferably 95% to 100% of the cells in the cell population are FOXA2+ imDAP cells. In some embodiments, at least about 60%, preferably from about 60% to about 90%, more preferably about 70% to about 90%, and most preferably 80% to 90% of the cells in the cell population are FOXA2+OTX2+ imDAP cells. In some embodiments, at least about 50%, preferably from about 50% to about 90%, more preferably about 50% to about 80%, and most preferably 60% to 80% of the cells in the cell population are LMX1A+ imDAP cells. In some embodiments, after enrichment, at least about 90%, preferably from about 95%, more preferably at least about 98% and most preferably 100% of the cells in the cell population obtained are LMX1A+ imDAP cells. In some embodiments, after enrichment, at least about 90%, preferably from about 95%, more preferably at least about 98% and most preferably 100% of the cells in the cell population obtained are OTX2+ imDAP cells. In some embodiments, the imDAP cells are FOXA2+OTX2+EN1+LMX1A+PAX6−NKX2.1− cells.

In still another aspect, the present disclosure provides a cell population comprising the imDAP cells or population thereof described herein.

In still further aspect, the present disclosure provides a pharmaceutical composition comprising any of the imDAP cells or cell populations thereof described herein and a pharmaceutically acceptable carrier. The amount of cell population used in the pharmaceutical composition that is effective in the treatment of a particular disorder or condition can depend on the nature of the disorder or condition and can be determined by standard clinical techniques.

Pharmaceutically acceptable carriers are well known in the art. Exemplary pharmaceutically acceptable carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, and other solutes. Non-limiting examples of such pharmaceutically acceptable carriers comprise Multiple Electrolytes Injection, and Dextran injection.

### Use

The imDAP cells or cell populations thereof described herein can be further matured to generate DA neurons. Further, the imDAP cells or cell populations thereof described herein have the capability to further mature and differentiate into DA neurons in vivo or in vitro. Accordingly, the imDAP cells or cell populations thereof described herein are applicable for cell therapy (e.g., cell transplantation) to treat or prevent diseases or conditions such as Parkinson's disease (PD).

In a further aspect, the present disclosure also provides a use of any of the imDAP cells or cell populations thereof described herein in the manufacture of a medicament for treating or preventing Parkinson's disease (PD).

In a further another aspect, the present disclosure provides a method of treating or preventing Parkinson's disease (PD), comprising administrating any of the imDAP cells or cell populations or pharmaceutical compositions thereof described herein to a subject in need thereof. Pharmaceutical compositions, cell compositions or populations of the present disclosure can be administered before, during, and/or after the onset of the disease, disorder, and/or condition.

### General Methods

In practicing the present disclosure, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984); Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

### EXAMPLES

The present description is further illustrated by the following examples, which should not be construed as limiting in any way. The contents of all cited references (including literature references, issued patents, and published patent applications as cited throughout this application) are hereby expressly incorporated by reference.

### Materials and Methods

All reagents for the culture media and apparatuses utilized throughout the Examples of the present disclosure are commercially available. The sources of these reagents and apparatuses have been also described elsewhere herein. The steps of the methods utilized throughout the Examples are described with reference to FIG. 1. The general testing methods utilized throughout the Examples are described as follows.

### General Testing Methods

(1) qRT-PCR
1. For qRT-PCR analysis of mRNA gene expression, RNA should be harvested from at least 2×10⁶ cells.
2. Harvest cells, add 350 μl of buffer RLT+1% (vol/vol) β-mercaptoethanol to the well, transfer the lysate to a 1.5 ml tube, and put it immediately on ice.
3. Perform RNA extraction using the Tiangen Kit (DP430) and following the manufacturer's protocol ‘Purification of total RNA from animal and human cells’.
4. When the RNA extraction was completed, determine the RNA concentration using a Nanodrop instrument.
5. Perform the first-strand cDNA synthesis reaction with 1 μg of RNA using the HiScript™ II Q RT SuperMIX for qPCR (+gDNA wiper) Kit (Vazyme, #R223-01) for RT-qPCR. Follow the manufacturer's ‘First-Strand cDNA Synthesis’ protocol. This reaction would yield a 20 μl product.
6. Dilute 10 μl cDNA product in 80 μl of nuclease-free water.
7. Pipette the diluted cDNA product (1 μl), Sybr green master mix (5 μl, Transgen, #AQ131) and 0.95 μM reverse/forward primer mix (4 μl) into a 384-well PCR plate in triplicate, either manually or using an automated pipetting robot. The complete primer panel included the target genes and the housekeeping gene (RPL13A). In the analysis, the expression level of gene was determined by using a sample of undifferentiated hPSCs as control.
8. Analyze the samples by running a quantitative PCR on the LightCycler 480 instrument using a 40x cycle two-step protocol with a 60° C., 60 sec annealing/elongation step and a 95° C., 30 sec denaturation step. The average CT value from the three technical replicates was used for calculation of relative gene expression using the ΔΔCT method. For each gene, calculate the FC of the differentiated samples relative to the undifferentiated control sample using the housekeeping gene for normalization. Next, calculate the average of the FC values on the basis of the housekeeping gene.

### (2) Immunocytochemical Analysis

### 1. Cell Fixation:

Remove the medium from the cells and wash with PBS. Add 4% (wt/vol) PFA (e.g., 200 μl to a well in a 48-well plate) and incubate at room temperature (RT) for 15 min. After incubation, wash the cells three times in PBS.

### 2. Blocking:

Remove PBS from the wells and add a blocking solution to the cells. Add enough volume to cover the cells. Leave the cells in the blocking solution for 1 h at RT.

### 3. Primary Antibody Incubation:

Remove the blocking solution from the cells and add a primary antibody solution (˜100 μl/cm²). Incubate for 1 h at RT on a shaker.

### 4. Secondary Antibody Incubation:

Remove the primary antibody solution and wash the cells three times in PBS. Add the secondary antibody solution to the cells (˜100 μl/cm²), wrap the plate in aluminum foil to avoid bleaching of the fluorophores and incubate for 1 h at RT on a shaker.

### 5. Dapi Incubation:

Remove the secondary antibody solution and wash the cells twice in PBS. Add the DAPI solution (1:1000) to the cells (˜100 μl/cm²), wrap the plate in aluminum foil to avoid bleaching of the fluorophores and incubate for 10 min at RT on a shaker.

6. Wash the cells three times in PBS and leave the plate wrapped in aluminum foil at 4° C. until analysis.
7. Analyze the immunocytochemically stained cells using a fluorescence microscope and estimate the number of positive cells.

### (3) Flow Cytometry Analysis

1. Individualize and collect cells by either Accutase or TrypLE treatment.
2. Wash cells with 1 ml PBS and pellet cells at 250×g for 15 sec.
3. Remove supernatant, add ˜200 μl of 4% PFA, mix and fix for 10 min at RT.
4. Pellet cells at 350×g for 5 min.
5. Wash cells by 1 ml of FACS buffer and pellet cells at 350×g for 5 min.
6. Remove supernatant, add 200 μl/tube of FACS buffer/0.1% Triton X, mix for 10 min at RT.
7. Wash cells by 1 ml of FACS buffer and pellet cells at 350×g for 5 min.
8. Remove supernatant, add 200 μl/tube of primary antibody diluted in FACS buffer, mix gently, and incubate for 30 min at RT.
9. Wash cells by 1 ml of FACS buffer and pellet cells at 350×g for 5 min.
10. Remove supernatant, and add 200 μl of secondary antibody diluted in FACS buffer, mix gently, and incubate for 30 min at RT.
11. Wash cells by 1 ml of FACS buffer and pellet cells at 300×g for 5 min.
12. Remove supernatant, add 200 μl of FACS buffer, and run samples on the flow cytometer.

### Example 1: Derivation of imDAP Cells from the 2-Step WNT Signaling Activation Protocol

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 10 μM SB431542, 1 μg/mL SHH C24II and 1.25 μM CHIR99021 and culturing the cells for 3 Days. From Day 3, the basal medium was changed to DMEM: F12/B27 (minus Vitamin A) (98:2, v/v). Then, SB431542 was withdrawn on Day 4, and SHH C24II was withdrawn on Day 8. From Day 10, imDAP cells were specified by changing the differentiation medium to a Stage 2-2 differentiation medium containing DMEM: F12/B27 (minus Vitamin A) (98:2, v/v) as basal medium and supplemented with 0.2 μM LDN193189, 100 ng/ml FGF8b and 3 μM CHIR99021 for 6 Days. During the cell culture, each of the differentiation media was replaced every day. On Day 16 (at the end of S2-2), imDAP cells were collected and analyzed by qRT-PCR assay for ventral midbrain marker EN1 and LMX1A. The above experiment was repeated for three times and the derived imDAP cells were named as Lot1, Lot2 and Lot3, respectively.

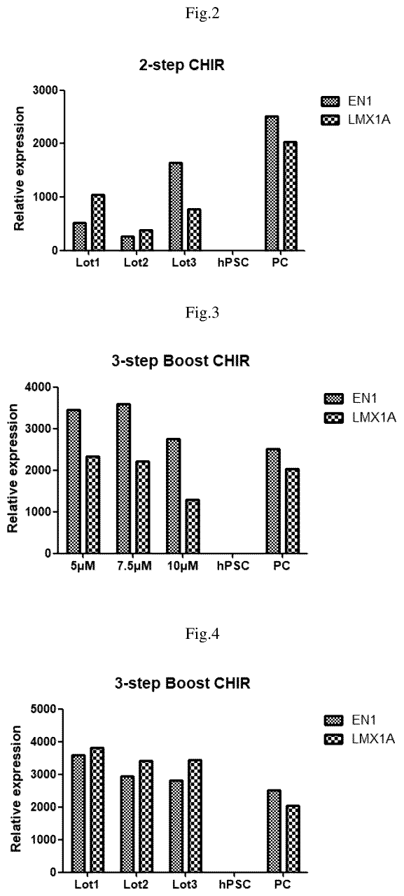

The results were shown in FIG. 2. In the above assay, the undifferentiated hiPSC (Nuwacell) was used as the negative control, and a plasmid (GenScript) containing the target sequence was used as the positive control (PC). Most of the previous imDAP derivation processes used 1-step or 2-step WNT signaling activation protocols to obtain correct midbrain fate while minimizing the contamination of diencephalic or hindbrain fates (Kirkeby et al., 2017; Kee et al., 2017; Studer et al., 2021). However, as demonstrated in the Example 1, it was found that 2-step WNT signaling protocol led to low expression levels of the midbrain markers EN1 and LMX1A and a significant batch-to-batch variability for derived imDAP cells, which shows a low efficiency for differentiation of hiPSCs into imDAP cells and an inferior consistency in the quality of different product batches.

### Example 2: Derivation of imDAP Cells from the 3-Step WNT Signaling Activation Protocol

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as a basal medium and supplemented with 0.2 μM LDN193189, 10 μM SB431542, 1 μg/mL SHH C24II and 1.25 μM CHIR99021 and culturing the cells for 3 Days. From Day 3, the basal medium was changed to DMEM: F12/B27 (minus Vitamin A) (98:2, v/v), and cells were exposed to high CHIR concentration of 5, 7.5 or 10 μM until Day 10 (Stage 2-2). SB431542 was withdrawn on Day 4, and SHH C24II was withdrawn on Day 8. From Day 10 (Stage 2-3), the differentiation medium was changed to a Stage 2-3 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-3), EBs were collected and analyzed by qRT-PCR assay for ventral midbrain marker EN1 and LMX1A.

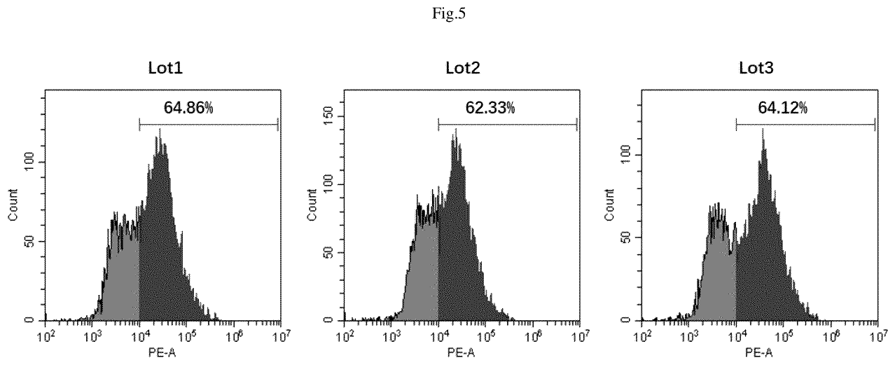

The results were shown in FIG. 3. In the above assay, the undifferentiated hiPSC (Nuwacell) was used as the negative control, and a plasmid (GenScript) containing the target sequence was used as the positive control (PC). As shown in FIG. 3, the expression levels of EN1 and LMX1A in the 3-step protocol with all three concentrations of CHIR were increased relative to the 2-step protocol described in Example 1 (FIG. 2), with 5 μM and 7.5 μM providing better results than 10 μM.

### Example 3: Stability of EN1 and LMX1A Expressions Among Different Batches of imDAP Cells Derived from the 3-Step WNT Signaling Activation Protocol

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 10 μM SB431542, 1 μg/mL SHH C24II and 1.25 μM CHIR99021 and culturing the cells for 3 Days. From Day 3, the basal medium was changed to DMEM: F12/B27 (minus Vitamin A) (98:2, v/v), and CHIR concentration was boosted to 7.5 μM until Day 10 (Stage 2-2). SB431542 was withdrawn on Day 4, and SHH C24II was withdrawn on Day 8. From Day 10 (Stage 2-3), the differentiation medium was changed to a Stage 2-3 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-3), imDAP cells were collected and analyzed by qRT-PCR assay for ventral midbrain marker EN1 and LMX1A. The above experiment was repeated for three times and the derived imDAP cells were named as Lot1, Lot2 and Lot3, respectively.

The results were shown in FIG. 4. In the above assay, the undifferentiated hiPSC (Nuwacell) was used as the negative control, and a plasmid (GenScript) containing the target sequence was used as the positive control (PC). As shown in FIG. 4, the expression levels of both EN1 and LMX1A in the 3-step protocol were high and consistent among the three batches, in contrast to the 2-step protocol described in Example 1 (shown in FIG. 2).

### Example 4: FOXA2 Expression for Different Batches of imDAP Cells Derived from the 3-Step WNT Signaling Activation Protocol

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 10 μM SB431542, 1 μg/mL SHH C24II and 1.25 μM CHIR99021 and culturing the cells for 3 Days. From Day 3, the basal medium was changed to DMEM: F12/B27 (minus Vitamin A) (98:2, v/v), and CHIR concentration was boosted to 7.5 μM until Day 10 (Stage 2-2). SB431542 was withdrawn on Day 4, and SHH C24II was withdrawn on Day 8. From Day 10 (Stage 2-3), the medium was changed to a differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-3), EBs were dissociated for single cells and collected for analysis. The cells of each group were stained with FOXA2-PE antibody (BD, #561689), the percentage of FOXA2+ imDAP cells were analyzed by flow cytometry (FIG. 5). The above experiment was repeated for three times and the derived imDAP cells were named as Lot1, Lot2 and Lot3, respectively.

As shown in FIG. 5, the percentages of cells positive for FOXA2 for Lot1, Lot2 and Lot3 in the 3-step protocol were as low as 64.86%, 62.33%, and 64.12%, respectively, and the cells exhibited good batch-to-batch consistency for the percentage of FOXA2+ cells.

### Example 5: Effect of Different Contact Time of High-Concentration CHIR on EN1 and LMX1A Expressions of imDAP Cells Derived from the 3-Step WNT Signaling Activation Protocol

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 10 μM SB431542, 1 μg/mL SHH C24II and 1.25 μM CHIR99021 and culturing the cells for 3 Days. From Day 3, the basal medium was changed to DMEM: F12/B27 (minus Vitamin A) (98:2, v/v), and CHIR concentration was boosted to 7.5 μM until Day 6 or Day 10 (Stage 2-2). SB431542 was withdrawn on Day 4, and SHH C24II was withdrawn on Day 8. From Day 10 (Stage 2-3), the medium was changed to a Stage 2-3 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-3), EBs were collected and analyzed by qRT-PCR assay for ventral midbrain marker EN1 and LMX1A.

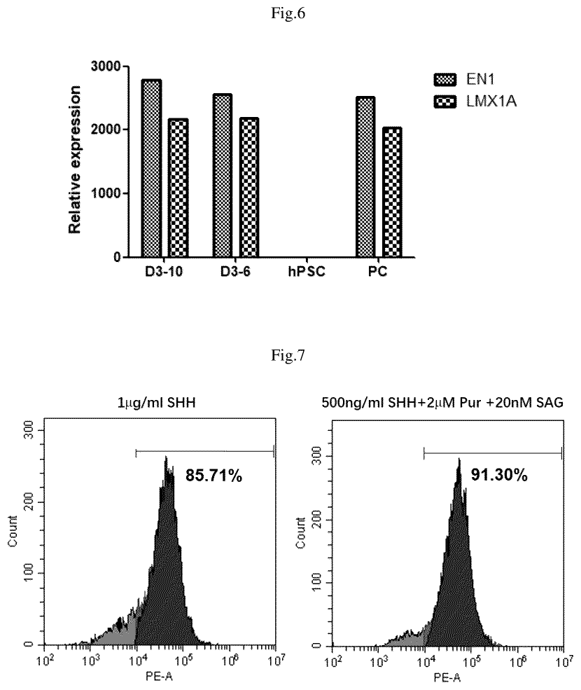

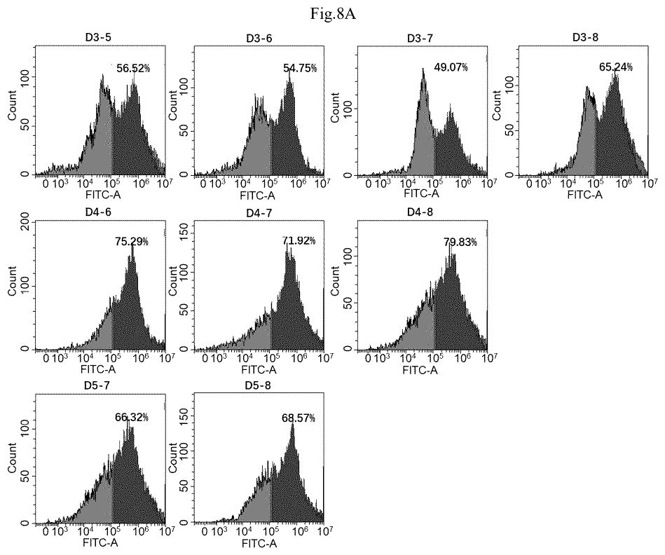

The results were shown in FIG. 6. In the above assay, the undifferentiated hiPSC (Nuwacell) was used as the negative control, and a plasmid (GenScript) containing the target sequence was used as the positive control (PC). As shown in FIG. 6, the expression levels of EN1 and LMX1A when the culture for Stage 2-2 was conducted from Day 3 to Day 6 were comparable to the expression levels of the markers when the culture for Stage 2-2 was conducted for a longer period of from Day 3 to Day 10.

As demonstrated in the examples 2-5, the 3-step WNT activation protocol showed reduced variability among different batches and increased expression levels of EN1 and LMX1A markers while avoiding both anterior (diencephalic) and posterior (hindbrain) contaminants as compared to the 2-step WNT activation protocol, however, the imDAP cells obtained from the 3-step WNT activation protocol were still inadequate in terms of the differentiation efficiency for FOXA2+ imDAP cells, which was not suitable for clinical applications.

### Example 6: Derivation of imDAP Cells from the 4-Step WNT Signaling Activation Protocol

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 20 μM SB431542, 1.2 μM CHIR99021, and 1 μg/mL SHH C24II or the combination of SHH agonists including 500 ng/mL SHH C24II, 2 μM Purmorphamine and 10 nM SAG and culturing the cells for 3 days. From Day 3, the basal medium was changed to DMEM: F12/B27 (minus Vitamin A) (98:2, v/v). Cells were exposed to Boost-CHIR concentration of 7.5 μM starting from Day 4 to Day 6 (Stage 2-2), followed by a low CHIR concentration of 1.2 μM (Stage 2-3) until Day 10. SB431542 was withdrawn on Day 6, and SHH C24II or the combination of SHH agonists was withdrawn on Day 10. From Day 10 (Stage 2-4), the medium was changed to a Stage 2-4 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-4), EBs were dissociated for single cells and collected for analysis. The cells of each group were stained with FOXA2-PE antibody (BD, #561689), the percentage of FOXA2+ imDAP cells were analyzed by flow cytometry (FIG. 7).

As shown in FIG. 7, the percentages of cells positive for FOXA2 were as high as 85.71% with SHH C24II as an SHH agonist, and 91.3% with the combination of SHH agonists including SHH C24II, Purmorphamine and SAG. These percentages as a result of the 4-step protocol in this Example were significantly increased as compared to the 3-step protocol described in Example 4 and shown in FIG. 5. Therefore, the 4-step protocol provided a greatly improved differentiation efficiency for FOXA2+ imDAP cells in contrast to the 3-step protocol.

### Example 7: Effect of Different Time Window for High-Concentration WNT Activator on OTX2 and PAX6 Expressions of Derived imDAP Cells in the 4-Step WNT Signaling Activation Protocol

### Experiment Procedure:

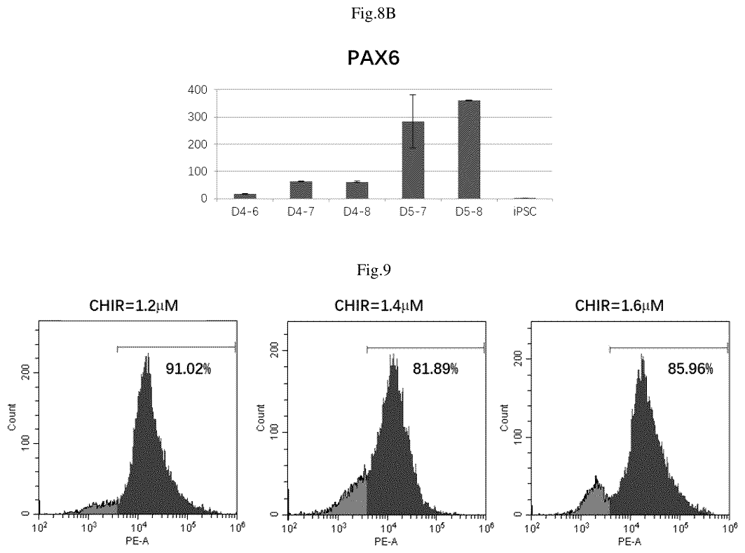

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.5 μM LDN193189, 10 μM SB431542, 500 ng/mL SHH C24II, 2 μM Purmorphamine, 10 nM SAG and 1.2 μM CHIR99021 and culturing the cells for 3 Days. From Day 3, the basal medium was changed to DMEM: F12/B27 (minus Vitamin A) (98:2, v/v), and cells were exposed to Boost-CHIR concentration of 7.5 μM starting from Day 3 to Day 5, Day 6, Day 7, or Day 8, from Day 4 to Day 6, Day 7 or Day 8, or from Day 5 to Day 7 or Day 8 (Stage 2-2). At Stage 2-3 (from Day 5, 6, 7 or 8 to Day 10), CHIR concentration was reduced to 1.2 μM. LDN193189 was withdrawn on Day 6, SB431542 was withdrawn on Day 8, and SHH C24II, Purmorphamine and SAG were withdrawn on Day 10. From Day 10 (Stage 2-4), the differentiation medium was changed to a Stage 2-4 differentiation medium containing DMEM: F12/B27 (plus Vitamin A) (98:2, v/v) as basal medium and supplemented with 3 μM CHIR99021. During the cell culture, each of the differentiation media was replaced every day. On Day 16 (at the end of S2-4), EBs were dissociated for single cells and collected for analysis. The cells of each group were stained with OTX2-FITC antibody (Miltenyi, #130-121-195), the percentage of OTX2+ imDAP cells were analyzed by flow cytometry (FIG. 8A). The cells obtained by Boost-CHIR treatment from D4 to D6, D7 or D8, or from D5 to D7 or D8 were analyzed by qRT-PCR for dorsal midbrain marker PAX6 (FIG. 8B).

As shown in FIG. 8A, exposure to Boost-CHIR concentration from Day 4 (Day 4 to Day 6, Day 4 to Day 7, and Day 4 to Day 8) in Stage 2-2 all achieved higher percentage of cells positive for OTX2 (75.29%, 71.92% and 79.83%, respectively) as compared to exposure to Boost-CHIR concentration from Day 3 or Day 5. In addition, as shown in FIG. 8B, the exposure to Boost-CHIR concentration from Day 4 also achieved cells lower in expression of PAX6 as compared to the exposure to Boost-CHIR concentration from Day 5. The above data showed that the exposure to Boost-CHIR concentration from Day 4 is more beneficial to the differentiation for imDAP cells.

### Example 8: FOXA2 Expression for imDAP Cells Derived from the 4-Step WNT Signaling Activation Protocol with Different Low Concentration of WNT Activator in Stage 2-3

### Experiment Procedure:

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 20 μM SB431542, 500 ng/mL SHH C24II, 2 μM Purmorphamine, 10 nM SAG and 1.2 μM CHIR99021 and culturing the cells for 3 Days. From Day 3, the basal medium was changed to DMEM: F12/B27 (minus Vitamin A) (98:2, v/v). Cells were exposed to Boost-CHIR concentration of 7.5 μM starting from Day 4 until Day 8 (Stage 2-2), followed by a low CHIR concentration of 1.2 μM, 1.4 μM and 1.6 μM from Day 8 until Day 10 (Stage 2-3). SB431542 was withdrawn on Day 8, and SHH C24II, Purmorphamine and SAG were withdrawn on Day 10. From Day 10 (Stage 2-4), the medium was changed to a Stage 2-4 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-4), EBs were dissociated for single cells and analyzed for FOXA2 expression by flow cytometry.

As shown in FIG. 9, all three low concentrations of CHIR in Stage 2-3 led to a high percentage of cells positive for FOXA2 (91.02%, 81.89%, and 85.96% for low CHIR concentrations of 1.2 μM, 1.4 μM and 1.6 μM, respectively, for Stage 2-3) and thus high differentiation efficiency for FOXA2+ imDAP cells.

### Example 9: Stability of FOXA2, OTX2, EN1 and LMX1A Expressions Among Different Batches of imDAP Cells Derived from the 4-Step WNT Signaling Activation Protocol

### Experiment Procedure:

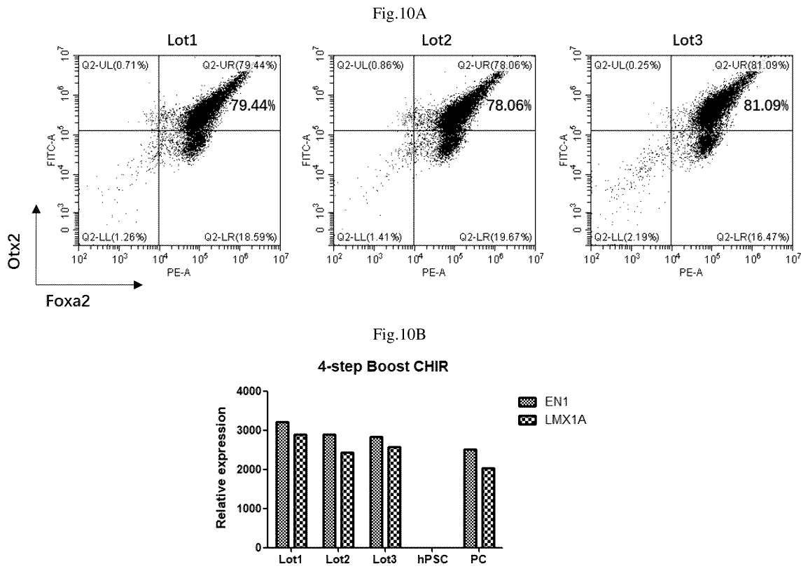

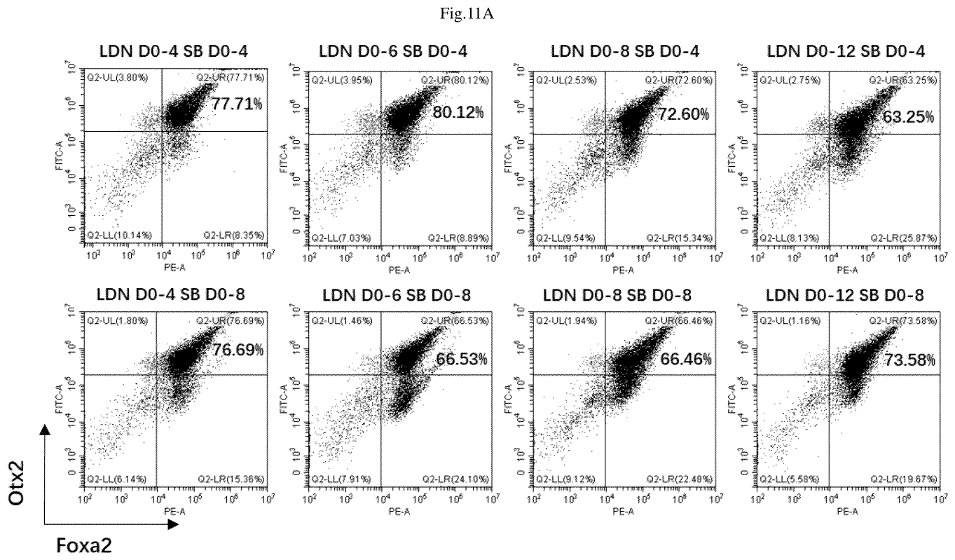

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing DMEM: F12/B27 (plus Vitamin A) (98:2, v/v) as basal medium and supplemented with 0.2 μM LDN193189, 20 μM SB431542, 500 ng/mL SHH C24II, 2 μM Purmorphamine, 10 nM SAG and 1.2 μM CHIR99021 and culturing the cells for 3 Days. From Day 3, the basal medium was changed to DMEM: F12/B27 (minus Vitamin A) (98:2, v/v). Cells were exposed to Boost-CHIR concentration of 7.5 μM starting from Day 4 until Day 8 (Stage 2-2), followed by a low CHIR concentration of 1.2 μM until Day 10 (Stage 2-3). SB431542 was withdrawn on Day 8, and SHH C24II, Purmorphamine and SAG were withdrawn on Day 10. From Day 10 (Stage 2-4), the medium was changed to a Stage 2-4 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 16 (at the end of S2-4), EBs were dissociated for single cells and collected for analysis. The above experiment was repeated for three times and the derived imDAP cells were named as Lot1, Lot2 and Lot3, respectively. The three independent bathes of cells (Lot1, Lot2, Lot3) were stained with FOXA2-PE antibody (BD, #561589) and OTX2-FITC antibody (Miltenyi, #130-121-195), the percentage of FOXA2+/OTX2+ imDAP cells were analyzed by flow cytometry (FIG. 10A) and the cells were also analyzed by qRT-PCR for ventral midbrain marker EN1 and LMX1A (FIG. 10B).

In the above assay, the undifferentiated hiPSC (Nuwacell) was used as the negative control, and a plasmid (GenScript) containing the target sequence was used as the positive control (PC). As shown in FIG. 10A, the percentages of cells positive for both FOXA2 and OTX2 (FOXA2+/OTX2+) from the three batches were as high as 79.44%, 78.06%, and 81.09%, respectively, and consistent from batch to batch. Similarly, the expression levels of EN1 and LMX1A were also high and consistent among the three batches. These imDAP cells with high expressions and high batch-to-batch consistency for all of FOXA2, OTX2, EN1 and LMX1A were very suitable for clinical applications.

### Example 10: Effect of Different Time Window for BMP and TGF-β Signaling Inhibition on the Specification of imDAP Cells

### Experiment Procedure:

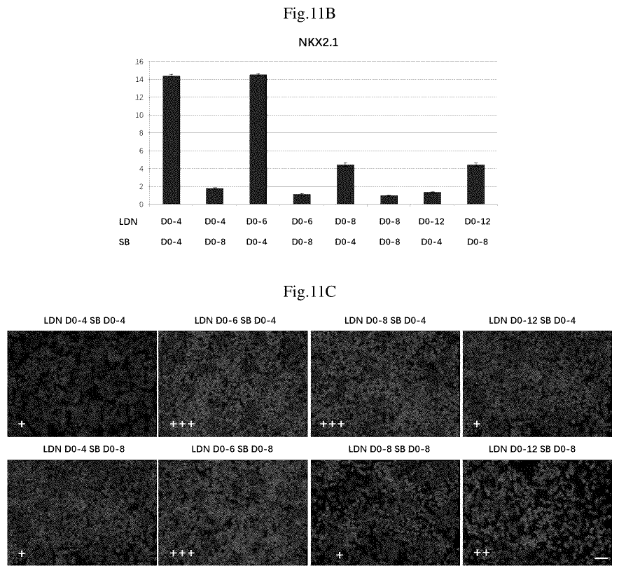

hiPSCs were cultured similarly to Example 9 to derive imDAP cells, except for the treatment time windows for LDN193189 and SB431542 as well as Stage 2-4. The treatment time window for LDN193189 was from Day 0 (D0) to Day 4 (D4), from Day 0 (D0) to Day 6 (D6), from Day 0 (D0) to Day 8 (D8), or from Day 0 (D0) to Day 12 (D12), and the treatment time window for SB431542 was from Day 0 (D0) to Day 4 (D4), or from Day 0 (D0) to Day 8 (D8). The time window for step 2-4 is from Day 10 to Day 12. On Day 12 (at the end of S2-4), EBs were dissociated for single cells and collected for analysis. The cells were stained with FOXA2-PE antibody (BD, #561589) and OTX2-FITC antibody (Miltenyi, #130-121-195), the percentage of FOXA2+/OTX2+ imDAP cells were analyzed by flow cytometry (FIG. 11A). The cells were also analyzed by qRT-PCR for diencephalic marker NKX2.1 (FIG. 11B) and were seeded on 48-well slide for immunostaining with LMX1A antibody (FIG. 11C).

As described above, the effect of the treatment window for the two inhibitors of SMAD signaling, that is, BMP and TGF signaling, on the specification of imDAP cells was systematically examined. Taking the results of FIGS. 11A-11C together, all the time windows tested provided the high percentages of the FOXA2+/OTX2+ imDAP cells while avoiding anterior (diencephalic) and posterior (hindbrain) contaminants, and treatment with LDN193189 from D0 to D6 and with SB431542 from D0 to D4 provided the best results among all the time windows tested.

### Example 11: Effect of Different Concentrations for BMP and TGFβ Signaling Inhibition on the Specification of imDAP Cells

### Experiment Procedure:

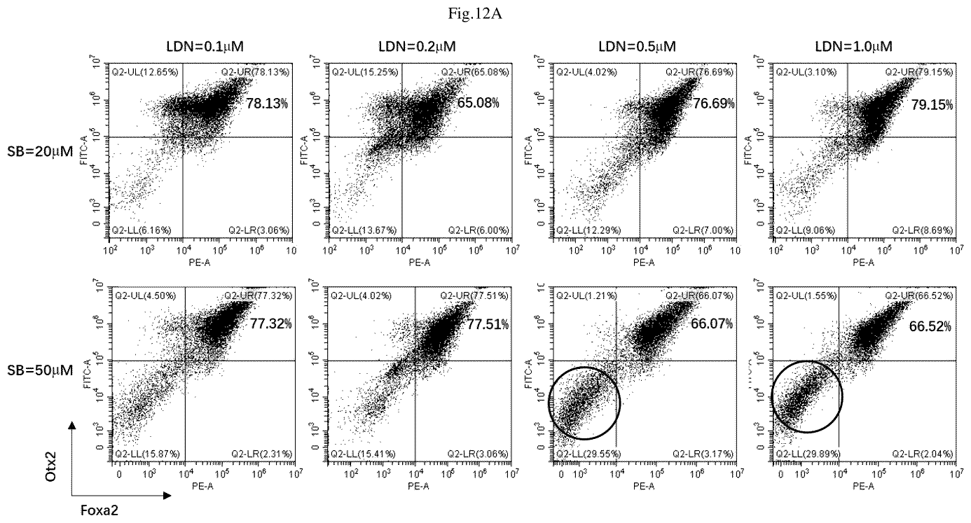

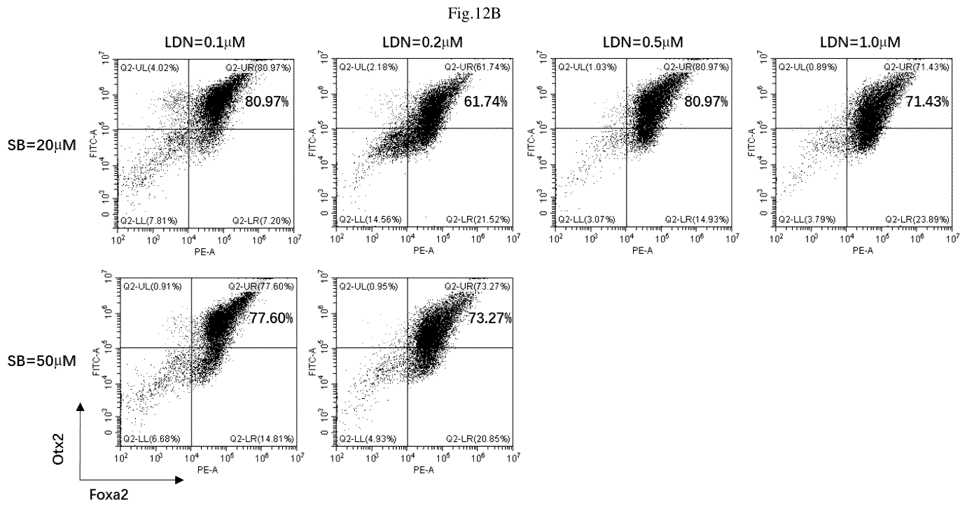

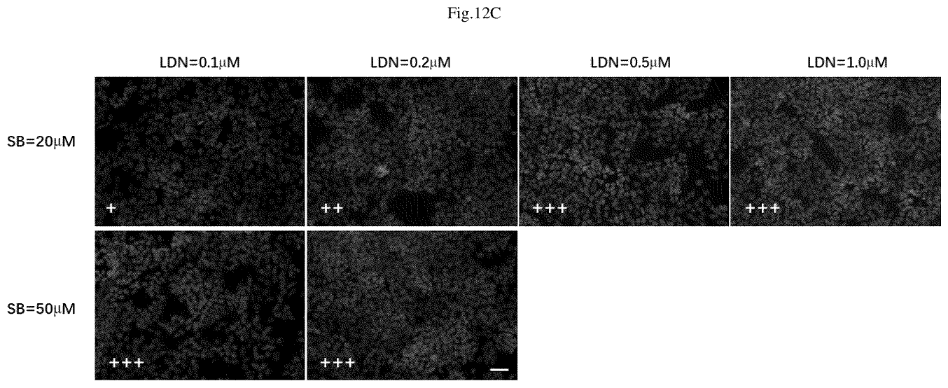

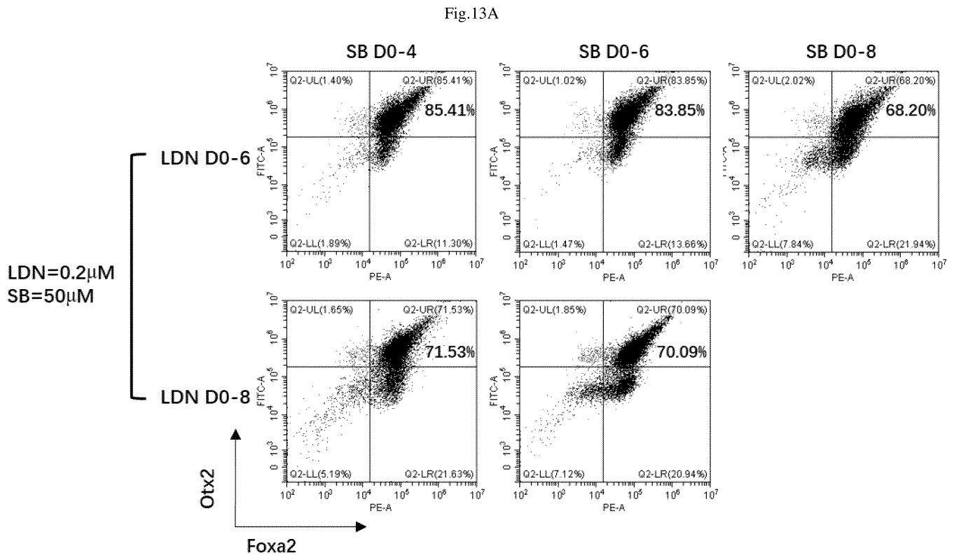

hiPSCs were cultured similarly to Example 9 to derive mDAPs. The concentration of LDN193189 was 0.1 μM, 0.2 μM, 0.5 μM or 1.0 μM, and the concentration of SB431542 was 20 μM or 50 μM. The time window for Stage 2-4 is from Day 10 to Day 12. On Day 6, some EBs were collected for FACS analysis of FOXA2 and OTX2 (FIG. 12A). On Day 12 (at the end of S2-4), EBs were dissociated for single cells and collected for FACS analysis of FOXA2 and OTX2 (FIG. 12B). In the FACS analysis, the cells were stained with FOXA2-PE antibody (BD, #561589) and OTX2-FITC antibody (Miltenyi, #130-121-195), and the percentage of FOXA2+/OTX2+ imDAP cells was analyzed by flow cytometry. The cells were also seeded on 48-well slide for immunostaining with LMX1A antibody (Millipore, #MAB10533) (FIG. 12C).

As described above, the effect of the concentrations for the two inhibitors of SMAD signaling, that is, BMP and TGF signaling, on the specification of imDAP cells was systematically examined. Taking the results of FIGS. 12A-12C together, treatment with LDN193189 at 0.5 μM and with SB431542 at 20 μM provided the best results among all the combinations of concentrations tested, and treatment with LDN193189 at 0.1 μM and SB431542 at 50 μM also provided similar superior results.

### Example 12: Effect of Different Combination of Concentration and Time Window for BMP and TGFβ Signaling Inhibition on the Specification of imDAP Cells

### Experiment Procedure:

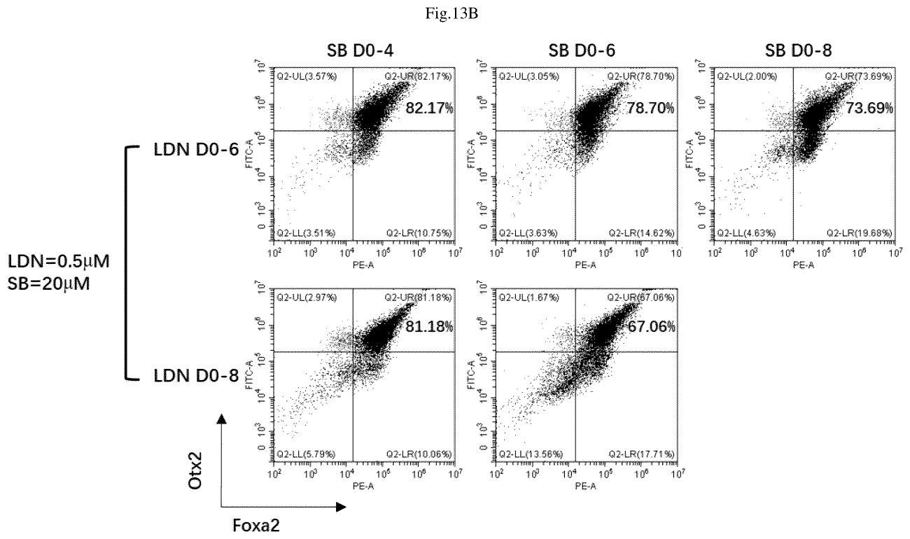

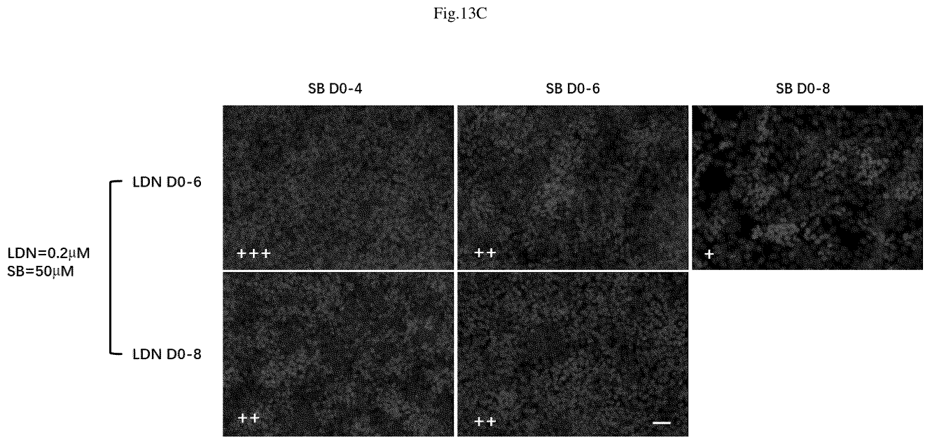

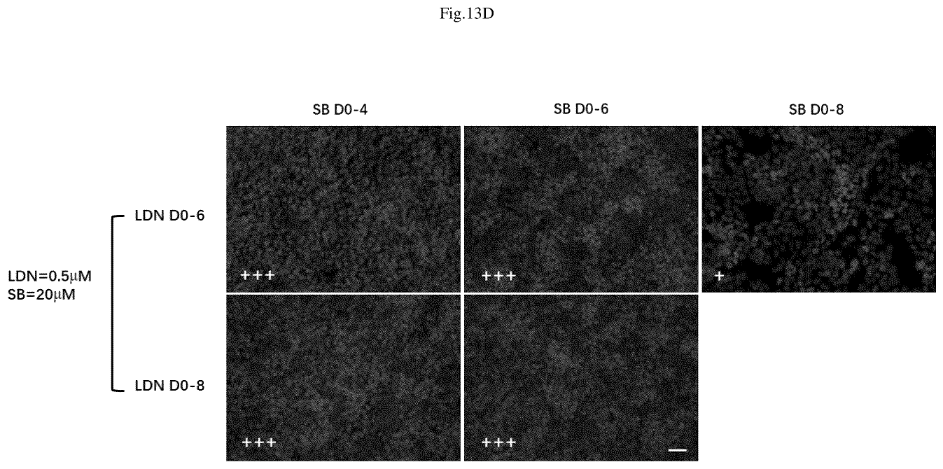

hiPSCs were cultured similarly to Example 9 to derive mDAPs. The concentrations of LDN193189 and SB431542 were set at 0.2 μM LDN193189 and 50 μM SB431542, or at 0.5 μM LDN193189 and 20 μM SB431542. The treatment time window for LDN193189 was from Day 0 (D0) to Day 6 (D6), or from Day 0 (D0) to Day 8 (D8), and the treatment time window for SB431542 was from Day 0 (D0) to Day 4 (D4), Day 0 (D0) to Day 6 (D6), or from Day 0 (D0) to Day 8 (D8). The time window for Stage 2-4 is from Day 10 to Day 12. On Day 12 (at the end of S2-4), EBs were dissociated for single cells and collected for analysis. The cells were stained with FOXA2-PE antibody (BD, #561589) and OTX2-FITC antibody (Miltenyi, #130-121-195), the percentage of FOXA2+/OTX2+ imDAP cells were analyzed by flow cytometry (FIGS. 13A and 13B). And the cells were also seeded on 48-well slides for immunostaining with LMX1A antibody (Millipore, #MAB10533) (FIGS. 13C and 13D).

As described above, the effects of the concentrations and time windows for the two inhibitors of SMAD signaling, that is, BMP and TGF signaling, on the specification of imDAP cells were systematically examined. Taking the results of FIGS. 13A-13D together, treatment with LDN193189 at 0.2 μM from Day 0 to Day 6 and with SB431542 at 50 μM from Day 0 to Day 4 or from Day 0 to Day 6 provided the best results among all the condition combinations tested, and treatment with LDN193189 at 0.5 μM from Day 0 to Day 6 or from Day 0 to Day 8 and with SB431542 at 20 μM from Day 0 to Day 4 also provided similar superior results.

### Example 13: Serum-Free and Xeno-Free Basal Medium Development for imDAP Differentiation

### Experiment Procedure:

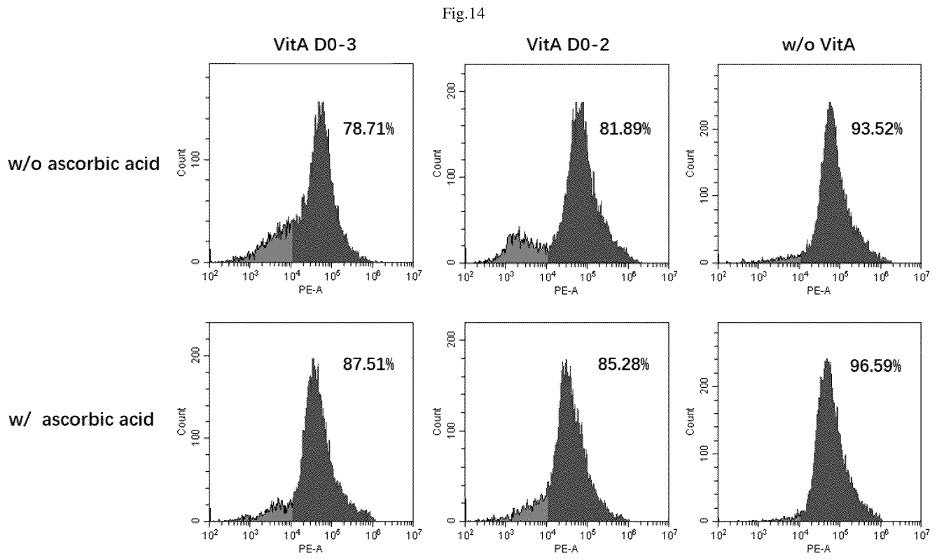

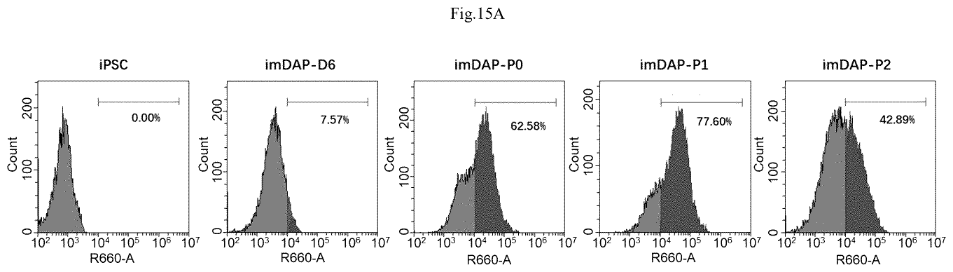

On Day-1, EB formation (Stage 1) was carried out by dissociating hiPSCs (Nuwacell) to form a single-cell suspension and resuspending it in Epic medium (Nuwacell, #RP01001) containing 10 μM Blebbistatin (MCE, #HY-13441) in T25 flasks on a belly dancer and culturing the cells overnight. On Day 0, ventral midbrain floor plate differentiation (Stage 2) was initiated by changing the medium to a Stage 2-1 differentiation medium containing an indicated basal medium and supplemented with 0.5 μM LDN193189, 20 μM SB431542, 500 ng/mL SHH C24II, 2 μM Purmorphamine, 10 nM SAG and 1.2 μM CHIR99021 and culturing the cells for 2 or 3 days. The basal medium used here was DMEM: F12/B27 (plus Vitamin A) (98:2, v/v) without L-ascorbic acid 2-phosphate sesquimagnesium salt (Aapss, Sigma-Aldrich, #A8960, CAS No.: 1713265-25-8), DMEM: F12/B27 (plus Vitamin A) (98:2, v/v) with 50 μg/mL of Aapss, DMEM: F12/B27 (minus Vitamin A) (98:2, v/v) without Aapss, or DMEM: F12/B27 (minus Vitamin A) (98:2, v/v) with 50 μg/mL of Aapss. From Day 2 or Day 3, the basal medium was changed to DMEM: F12/B27 (minus Vitamin A) (98:2, v/v) with 50 μg/mL of Aapss, or DMEM: F12/B27 (minus Vitamin A) (98:2, v/v) without Aapss. Cells were exposed to Boost-CHIR concentration of 7.5 μM starting from Day 4 until Day 8 (Stage 2-2), followed by a low CHIR concentration of 1.2 μM until Day 10 (Stage 2-3). LDN193189 was withdrawn on Day 6. SB431542 was withdrawn on Day 8. SHH C24II, Purmorphamine and SAG were withdrawn on Day 10. From Day 10 (Stage 2-4), the medium was changed to a Stage 2-4 differentiation medium containing DMEM: F12/B27 (minus Vitamin A) (98:2, v/v) as basal medium and supplemented with 0.2 μM LDN193189 and 3 μM CHIR99021. During the cell culture, each of the differentiation media was replaced every day. On Day 12 (at the end of S2-4), EBs were dissociated for single cells and collected for analysis. The cells were stained with FOXA2-PE antibody (BD, #561589), and the percentage of FOXA2+ imDAP cells were analyzed by flow cytometry (FIG. 14).

As shown in FIG. 14, by comparing the cases without Aapss (w/o ascorbic acid) to those with Aapss (w/ascorbic acid), addition of Aapss to the basal medium throughout the WNT signaling activation process led to higher percentage of FOXA2+ cells. Further, by comparing the cases with Vitamin A only during the initial days of differentiation (VitA D0-3 and VitA D0-2) to those without Vitamin A (w/o VitA) throughout the differentiation, the latter led to higher percentage of FOXA2+ cells. In particular, both omission of Vitamin A and addition of Aapss to the basal medium led to the highest percentage (96.59%) of FOXA2+ cells, and thus the optimal efficiency for differentiation.

### Example 14: Selection of Timing for imDAP Enrichment

### Experiment Procedure:

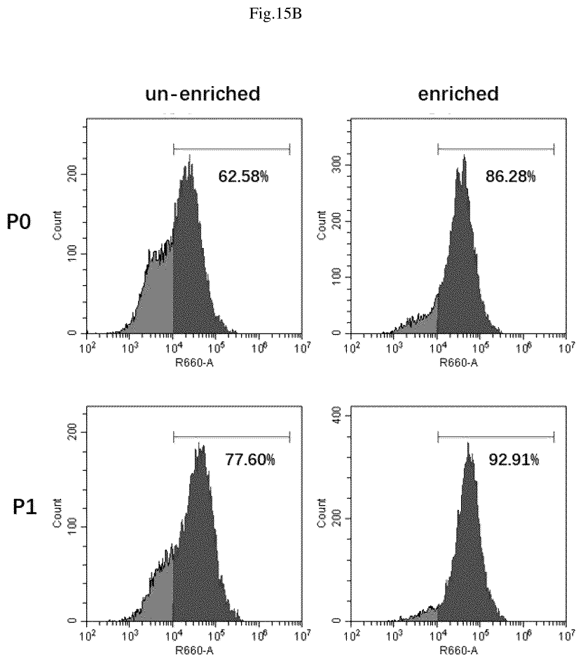

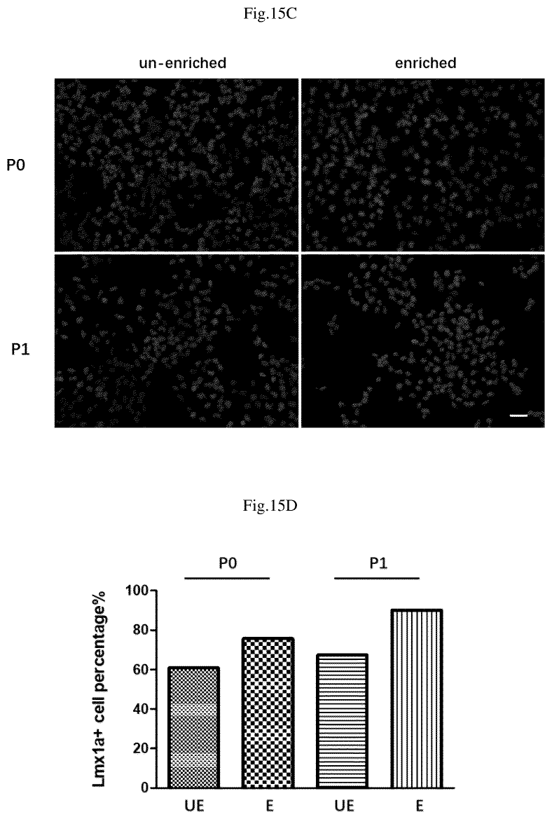

imDAP cells were collected on Day 6 and Day 12 (P0) during imDAP differentiation described in Example 12. The imDAP cells (P0) were seeded on DLL4/VTN-coated plates at a low density of 1×10⁴ cells/cm² in an expansion medium. The expansion medium used here contained the following components: DMEM: F12/B27 (99:1, v/v), 10 μM Y-27632, 0.1 μM LDN193189, 5 μM SB431542, and 3 μM CHIR99021. The expansion medium was replaced every 3rd day until reaching 100% confluence (6 days) for cell passaging. The imDAP cells were passaged enzymatically with 0.5× TrypLE (Gibco, #12563011), centrifuged at 250 g for 5 minutes, counted and plated on new DLL4/VTN-coated plates at a density of 1×10⁴ cells/cm² in the expansion medium. imDAP cells were expanded and maintained in the expansion medium for 2 passages (P1 and P2). The imDAP cells of P1 and P2 were collected. These cells collected on Day 6, Day 12 (P0), P1 and P2 were stained with TPBG-APC antibody (R&D System, #FAB49751A), the percentage of TPBG+ imDAP cells were analyzed by flow cytometry (FIG. 15A). According to the results of FIG. 15A, P0- and P1-imDAP cells were chosen to isolate TPBG+ cells via MACS. The single cells were stained with TPBG-APC antibody (R&D System, #FAB49751A) in a sorting buffer for 60 min at 4° C., and the labeled cells were incubated with APC-Microbeads (Miltenyi, #130-090-855) for 30 min at 4° C. The sorting buffer used here contained the following components: 1×DPBS (95%), 5% HSA, 10 μM Y-27632, and 0.5 μM EDTA. TPBG+ cells were separated from TPBG-cells using a separation column (LS column) following the manufacturer's instructions. The percentage of TPBG+ cells before and after sorting were shown in FIG. 15B. The unenriched and enriched imDAP cells at P0 and P1 were also seeded on 48-well slides for immunostaining with LMX1A antibody (FIGS. 15C and 15D).

In the above assay, the undifferentiated hiPSC (Nuwacell) was used as the negative control. It was found here that the TPBG and LMX1A expressions showed positive correlation within a narrow time window during directed differentiation and expansion. As shown in FIG. 15A, the cells of P0 and P1 (imDAP-P0 and imDAP-P1), and in particular the cells of P1, were much higher than the cells differentiated for 6 days (imDAP-D6) and the cells of P2 (imDAP-P2) in terms of the expression of TPBG. FIG. 15B showed the high percentages of TPBG+ cells for the cells of P0 and P1 before and after enrichment. The results in FIGS. 15B, 15C and 15D showed the high correlation between the TPBG+ cells and the LMX1A+ cells. The above data showed that selection of the cells of P0 or P1 for enrichment was very beneficial for efficient sorting of LMX1A+ imDAP cells.

### Example 15: Effect of Different Seeding Density During the Expansion of P0 imDAP Cells on Cell Sorting

### Experiment Procedure:

The imDAP cells (P0) produced in Example 12 was cryopreserved for a period. The process for cryopreservation was as follows. On Day 12 (at the end of S2-4), EBs were harvested and wash with DPBS (w/o Ca²⁺/Mg²⁺) to get rid of dead and floating cells. Add 5 ml/flask of 2x TrypLE. Plate the flask on belly dancer at 37° C. for 10-13 min to ensure that all EBs were dissociated completely into single cells. When the EBs were dissociated, transfer the cell suspension to the 50 ml tube and add 40 ml DPBS (w/o Ca²⁺/Mg²⁺). Spin down the cells at 300 g for 5 min at RT. After spinning, aspirate the medium without disturbing the cell pellet, resuspend the pellet with freezing medium-A (Nuwacell, Cat. No. 070083) to get an estimated cell concentration of about 1.2-2×10⁷ cells per ml. Count the cells using an automated cell counter (Countstar), and adjust the cell concentration to 1.2×10⁷ cells per ml. Then add equal volume of freezing medium-B (Nuwacell, Cat. No. 070084) to get the final cell concentration of 6×10⁶ cells per ml. Aliquot 1 ml of cell suspension per cryotube. Put the cryotubes into the precooled freezing container and transfer immediately to −80° C. Transfer the cryotubes containing the cells to liquid nitrogen in the following morning.

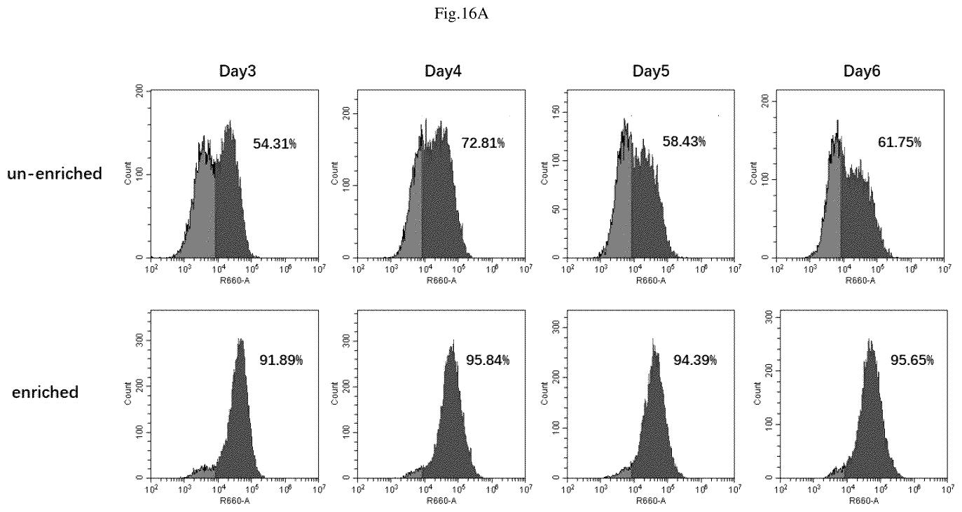

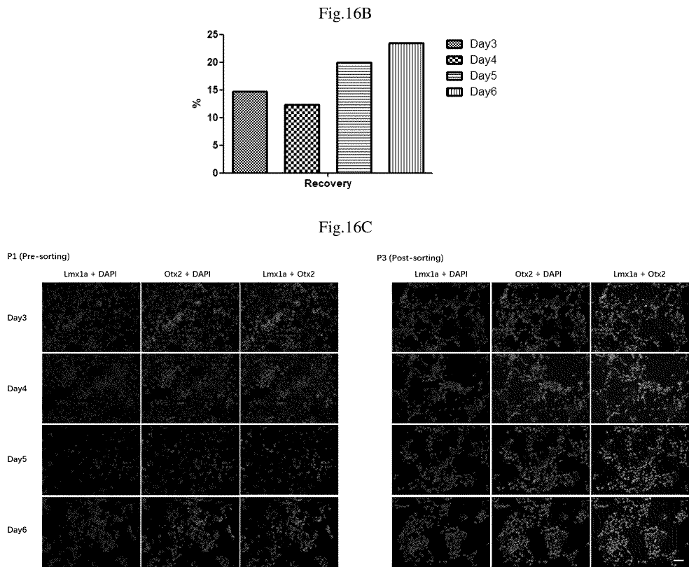

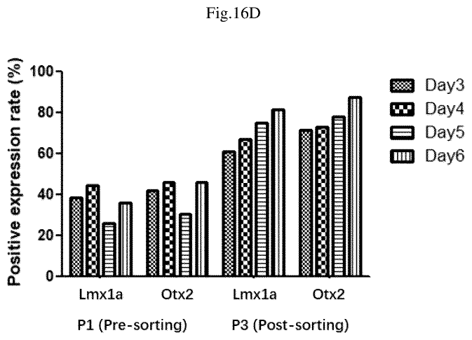

The cryopreserved cells were thawed. The process for thawing was as follows. Bring the cells to the cell culture lab on dry ice. Spray the cryotube with ethanol, and thaw it in a 37° C. water bath. When thawing was complete, spray the tube with ethanol and wipe it off before taking it into the cell culture hood. Transfer the cell suspension to an empty 15 ml tube, and add 10 ml of wash medium dropwise to the cells. Spin down cells at 300 g for 5 min at RT. Aspirate the medium from the cells and resuspend them with an expansion medium. The thawed cells were seeded on DLL4/VTN coated plates at the density of 8×10⁴/cm², 4×10⁴/cm², 2×10⁴/cm² and 1×10⁴/cm² in the expansion medium, respectively. The expansion medium used here contained the following components: DMEM: F12/B27 (99:1, v/v), 10 μM Y-27632, 0.1 μM LDN193189, 5 μM SB431542, and 3 μM CHIR99021. The cells were dissociated enzymatically with 0.5× TrypLE (Gibco, #12563011) until reaching 100% confluence (3 days, 4 days, 5 days and 6 days, respectively). To examine the sorting efficiency at different seeding density during expansion, the single cells collected on Day 3, Day 4, Day 5 and Day 6 were stained with TPBG-APC antibody (R&D System, #FAB49751A) in a sorting buffer for 60 min at 4° C., and the labeled cells were incubated with APC-Microbeads (Miltenyi, #130-090-855) for 30 min at 4° C. The sorting buffer used here contained the following components: 1×DPBS (95%), 5% HSA, 10UM Y-27632, and 0.5 μM EDTA. TPBG+ cells were separated from TPBG-cells using a separation column (LS column) following the manufacturer's instructions. The percentage of TPBG+ cells before and after sorting were shown in FIG. 16A, and the sorting recovery was shown in FIG. 16B. The enriched P1-imDAP cells are expanded under the above same conditions to obtain P3-imDAP cells (post-sorting). The P1-imDAP cells (pre-sorting) and P3-imDAP cells (post-sorting) in each group were seeded on 48-well slides for immunostaining with LMX1A antibody (Millipore, #FAB10533) and OTX2 antibody (R&D System, #AF1979) (FIGS. 16C and 16D). Sorting recovery=(Enriched TPBG+ cell number/Total cells)×100%.

FIG. 16A showed the percentages of the TPBG+ cells of P1 obtained by expansion at different seeding density before and after enrichment. As shown in FIG. 16A, different seeding density affected the percentage of TPBG+ cells before enrichment, and the percentage of TPBG+ cells (shown by Day 4) obtained at a density of 4×10⁴/cm² was the highest before enrichment, but the percentages of TPBG+ cells obtained under all seeding densities were similar after enrichment. Further, as shown in FIG. 16B, expansion at a lower seeding density of 1×10⁴/cm² (shown by Day 6) or 2×10⁴/cm² (shown by Day 5) provided higher sorting recovery for the TPBG+ cells than expansion at a higher seeding density of 4×10⁴/cm² (shown by Day 4) or 8×10⁴/cm² (shown by Day 3). In particular, expansion at a density of 1×10⁴/cm² provided the best sorting recovery for the TPBG+ cells. Similarly, as shown in FIGS. 16C-16D, expansion at a lower seeding density of 1×10⁴/cm² or 2×10⁴/cm² provided more efficient enrichment for the LMX1A+ cells and OTX2+ cells than expansion at a higher seeding density of 4×10⁴/cm² or 8×10⁴/cm². The results showed that selection of a low seeding density during expansion led to more efficient imDAP enrichment.

One skilled in the art would readily appreciate that the methods, compositions, and products described herein are representative of exemplary embodiments, and not intended as limitations on the scope of the disclosure. It will be readily apparent to one skilled in the art that varying substitutions and modifications may be made to the present disclosure disclosed herein without departing from the scope and spirit of the disclosure.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure is not to be limited in terms of the particular embodiments described in this application, which are intended as single illustrations of individual aspects of the disclosure. All the various embodiments of the present disclosure will not be described herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

1. A method for producing induced midbrain dopaminergic progenitor (imDAP) cells, comprising:
culturing pluripotent stem cells (PSCs) to form embryonic bodies (EBs); and
differentiating the EBs into imDAP cells by a multistep WNT signaling activation process, thereby obtaining a cell population comprising imDAP cells,
wherein the multistep WNT signaling activation process comprises:
(i) contacting the EBs with a first differentiation medium comprising a WNT signaling pathway activator at a first concentration,
(ii) contacting the cells from step (i) with a second differentiation medium comprising the WNT signaling pathway activator at a second concentration,
(iii) contacting the cells from step (ii) with a third differentiation medium comprising the WNT signaling pathway activator at a third concentration, and
(iv) contacting the cells from step (iii) with a fourth differentiation medium comprising the WNT signaling pathway activator at a fourth concentration,
wherein the first and third concentrations are lower than the fourth concentration, and the second concentration is higher than the fourth concentration.

2. The method of claim 1, wherein the first and third concentrations of the WNT signaling pathway activator are each independently in the range from about 0.2 μM to about 1.6 μM.

3. The method of claim 1, wherein the second concentration of the WNT signaling pathway activator is in the range from about 4 μM to about 10 μM.

4. The method of claim 1, wherein the fourth concentration of the WNT signaling pathway activator is in the range from about 2 μM to about 3.5 μM.

5. The method of claim 1, wherein the WNT signaling pathway activator in each of the first to fourth differentiation media comprises a glycogen synthase kinase-3 (GSK3) inhibitor, e.g., CHIR99021.

6. The method of claim 1, wherein the first differentiation medium further comprises a BMP4 inhibitor, a TGF-β inhibitor, and a sonic hedgehog agonist, and the second to fourth differentiation media each further comprise none, one or more of a BMP4 inhibitor, a TGF-β inhibitor, and a sonic hedgehog agonist.

7. The method of claim 6, wherein the concentration of the BMP4 inhibitor, if present in the first, second, third, or fourth differentiation medium, is in the range from about 0.05 μM to about 2.0 μM.

8. The method of claim 6, wherein the BMP4 inhibitor is Noggin, Chordin, Follostatin, Dorsomorphin, LDN193189, or any combination thereof.

9. The method of claim 6, wherein the concentration of the TGF-β inhibitor, if present in the first, second, third, or fourth differentiation medium, is in the range from about 1 μM to about 100 μM.

10. The method of claim 6, wherein the TGF-β inhibitor is RepSox, A83-01, SB431542, D4476, GW788388, LY364947, LY580276, SB525334, SB505124, SD208, GW6604, SJN-2511, or any combination thereof.

11. The method of claim 6, wherein the concentration of the sonic hedgehog agonist, if present in the first, second, third, or fourth differentiation medium, is in the range from about 100 ng/ml to about 2000 ng/mL.

12. The method of claim 6, wherein the sonic hedgehog agonist is SHH, SHH C24II, SHH C25II, SAG, SMO-IN-1, purmorphamine, a derivative thereof, or any combination thereof.

13. The method of claim 6, wherein the first differentiation medium comprises CHIR99021, LDN193189, SB431542, and SHH C24II, and the second to fourth differentiation media each comprise CHIR99021 and none, one or more of LDN193189, SB431542, and SHH C24II.

14. The method of claim 6, wherein the first differentiation medium comprises CHIR99021, LDN193189, SB431542, SHH C24II, Purmorphamine and SAG, and the second to fourth differentiation media each comprise CHIR99021 and none, one or more of LDN193189, SB431542, SHH C24II, Purmorphamine, and SAG.

15. The method of claim 1, wherein the first to fourth differentiation media further comprise a serum-free and xeno-free neural basal medium.

16. The method of claim 15, wherein the neural basal medium in the first to fourth differentiation media comprises an ascorbic compound, e.g., L-ascorbic acid 2-phosphate sesquimagnesium salt.

17. The method of claim 15, wherein the neural basal medium is free of vitamin A.

18. The method of claim 16, wherein the concentrations of the ascorbic compound in the first to fourth differentiation media each are about 5 μg/mL to about 100 μg/mL.

19. The method of claim 1, wherein the duration for step (i) is about 3 to about 4 days.

20. The method of claim 1, wherein the duration for step (ii) is about 2 to about 5 days.

21. The method of claim 1, wherein the duration for step (i) is about 4 days and the duration for step (ii) is about 2 to about 4 days.

22. The method of claim 1, wherein the duration for step (iii) is about 2 to about 5 days.

23. The method of claim 1, wherein the duration for step (iv) is about 2 to about 6 days.

24. The method of claim 1, wherein the time window for performing the step (i) is from Day 0 to Day 4 and the time window for performing the step (ii) is from Day 4 to Day 6, from Day 4 to Day 7, or from Day 4 to Day 8.

25. The method of claim 24, wherein the time window for performing the step (i) is from Day 0 to Day 4 and the time window for performing the step (ii) is from Day 4 to Day 8.

26. The method of claim 6, wherein the timing for treatment of the BMP inhibitor in the multistep WNT signaling activation process is from Day 0 to Day 6 and the timing for treatment of the TGF-β inhibitor in the multistep WNT signaling activation process is from Day 0 to Day 4.

27. The method of claim 6, wherein the concentration of the BMP inhibitor is 0.5 μM, and the concentration of the TGF-β inhibitor is 20 μM.

28. The method of claim 6, wherein the concentration of the BMP inhibitor is 0.1 μM, and the concentration of the TGF-β inhibitor is 50 μM.

29. The method of claim 6, wherein the concentration of the BMP inhibitor is 0.2 μM, the concentration of the TGF-β inhibitor is 50 μM, the timing for treatment of the BMP inhibitor in the multistep WNT signaling activation process is from Day 0 to Day 6, and the timing for treatment of the TGF-β inhibitor in the multistep WNT signaling activation process is from Day 0 to Day 4, or from Day 0 to Day 6.

30. The method of claim 13, wherein the concentration of the BMP inhibitor is 0.5 μM, the concentration of the TGF-β inhibitor is 20 μM, the timing for treatment of the BMP inhibitor in the multistep WNT signaling activation process is from Day 0 to Day 6, or from Day 0 to Day 8, and the timing for treatment of the TGF-β inhibitor in the multistep WNT signaling activation process is from Day 0 to Day 4.

31. The method of claim 1, further comprising expanding the imDAP cells after differentiation.

32. The method of claim 31, wherein the imDAP cells are expanded for one passage after differentiation.

33. The method of claim 32, wherein the imDAP cells are expanded at a seeding density of about 1×10⁴ cells/cm² to about 3×10⁴ cells/cm².

34. The method of according to claim 1, further comprising enriching the imDAP cells by sorting for TPBG+ cells after differentiation.

35. The method of according to claim 32, further comprising enriching the imDAP cells by sorting for TPBG+ cells after expansion for one passage.

36. The method of claim 1, wherein, after differentiation, at least about 78%, preferably at least about 85%, more preferably at least 90%, and most preferably 95% to 100% of the cells in the cell population without any enrichment are FOXA2+ imDAP cells.

37. The method of claim 1, wherein, after differentiation, at least about 60%, preferably from about 60% to about 90%, more preferably about 70% to about 90%, and most preferably 80% to 90% of the cells in the cell population without any enrichment are FOXA2+OTX2+ imDAP cells.

38. The method of claim 1, wherein, after differentiation, at least about 50%, preferably from about 50% to about 90%, more preferably about 50% to about 80%, and most preferably 60% to 80% of the cells in the cell population without any enrichment are LMX1A+ imDAP cells.

39. The method of claim 35, wherein after enrichment, at least about 90%, preferably from about 95%, more preferably at least about 98% and most preferably 100% of the cells in the cell population obtained are LMX1A+ imDAP cells.

40. The method of claim 35, wherein after enrichment, at least about 90%, preferably from about 95%, more preferably at least about 98% and most preferably 100% of the cells in the cell population obtained are OTX2+ imDAP cells.

41. The method of claim 1, wherein the imDAP cells are FOXA2+OTX2+EN1+LMX1A+PAX6−NKX2.1− cells.

42. A population of imDAP cells prepared by the method according to claim 1.
